(19) United States
(12) Patent Application Publication
(10) Pub. No.: US 2025/0288662 A1
(43) Pub. Date: Sep. 18, 2025

(54) MULTIVALENT DISPLAY ON ENVELOPED PARTICLES WITH HUMAN OLIGOMERIZATION DOMAINS

(71) Applicant: Achelois BioPharma, Inc., Redwood City, CA (US)

(72) Inventors: Michael CHEN, Palo Alto, CA (US); Chang-Zheng CHEN, Palo Alto, CA (US)

(21) Appl. No.: 18/861,302

(22) PCT Filed: Apr. 28, 2023
(86) PCT No.: PCT/US2023/066369

§ 371 Date: Oct. 29, 2024

Related U.S. Application Data
(60) Provisional application No. 63/338,177, filed on May 4, 2022.

(51) Int. Cl. A61K 39/215; A61K 39/00; A61P 31/14; C07K 14/005; C07K 14/52; C07K 14/525; C07K 14/565; C07K 14/57; C07K 14/705; C07K 14/715; C07K 14/75; C07K 14/785; C12N 9/24; C12N 9/48; C12N 15/86

(52) U.S. Cl. CPC A61K 39/215; A61P 31/14; C07K 14/005; C07K 14/522; C07K 14/5255; C07K 14/565; C07K 14/57; C07K 14/70521; C07K 14/70525; C07K 14/70532; C07K 14/70578; C07K 14/7158; C07K 14/75; C07K 14/785; C12N 9/2402; C12N 9/485; C12N 15/86; A61K 2039/5258; A61K 2039/70; C07K 2319/02; C07K 2319/03; C07K 2319/73; C07K 2319/74; C12N 2710/10041; C12N 2740/15043; C12N 2750/14143; C12N 2760/16222; C12N 2760/16223; C12N 2760/16234; C12N 2760/20222; C12N 2760/20223; C12N 2760/20234; C12N 2770/00; C12N 2770/20022; C12N 2770/20034; C12N 2770/24122; C12N 2770/24123; C12N 2770/24134; C12Y 302/01018; C12Y 304/15001

(57) ABSTRACT

Disclosed herein are recombinant fusion protein comprising transmembrane domains, display polypeptides, and human oligomerization domains, and enveloped particles containing the same, and methods of use.

### CROSS REFERENCE

This application claims the benefit of U.S. Provisional Application No. 63/338,177, filed May 4, 2022, which is incorporated herein by reference in its entirety.

### SEQUENCE LISTING

The instant application contains a Sequence Listing in electronic format. The Sequence Listing is provided as a file entitled 48295-711_601_SL.xml, created on Apr. 26, 2023, which is 116,702 bytes in size. The information in the electronic format of the Sequence Listing is incorporated by reference in its entirety.

### BRIEF SUMMARY

Disclosed herein, in certain embodiments, are a recombinant fusion protein comprising a transmembrane domain, a display polypeptide, and a human oligomerization domain, wherein when the recombinant fusion protein is expressed on a surface of an enveloped particle, the recombinant fusion protein is displayed in an oligomeric format. In some embodiments, the human oligomerization domain comprises a coiled coil motif. In some embodiments, the human oligomerization domain is a dimerization domain. In some embodiments, the human oligomerization domain comprises a human fibrinogen-like protein 1 coiled coil (hFcc) domain. In some embodiments, the human oligomerization domain is a trimerization domain. In some embodiments, the human oligomerization domain comprises a LINE-1 retrotransposon orf1p coiled coil (L1cc) domain. In some embodiments, the human oligomerization domain comprises a human lung surfactant protein D coiled coil (hLSPDcc) domain. In some embodiments, the human oligomerization domain comprises a human tetranectin coiled coil (hTETNcc) domain. In some embodiments, the human oligomerization domain is a tetramerization domain. In some embodiments, the human oligomerization domain comprises a human guanylate-binding protein 1 coiled coil (hGBP1cc) domain. In some embodiments, the human oligomerization domain comprises an amino acid sequence that has at least 85% sequence identity to an amino acid sequence according to SEQ ID NOs: 1-6. In some embodiments, the human oligomerization domain comprises an amino acid sequence that has at least 95% sequence identity to an amino acid sequence according to SEQ ID NOs: 1-6.

In some embodiments, when the recombinant fusion protein is expressed on the surface of the enveloped particle, the human oligomerization domain is outside of the enveloped particle. In some embodiments, when the recombinant fusion protein is expressed on the surface of the enveloped particle, the human oligomerization domain is outside of the enveloped particle and adjacent to a signal peptide. In some embodiments, when the recombinant fusion protein is expressed on the surface of the enveloped particle, the human oligomerization domain is inside of the enveloped particle. In some embodiments, when the recombinant fusion protein is expressed on the surface of the enveloped particle, and wherein the human oligomerization domain is inside of the enveloped particle and adjacent to the transmembrane domain.

In some embodiments, the recombinant fusion protein further comprises a signal peptide. In some embodiments, domains of the recombinant fusion protein are arranged from the N-terminus to the C-terminus in the following orders: a) signal peptide, display polypeptide, human oligomerization domain, and transmembrane domain; b) signal peptide, display polypeptide, transmembrane domain, and human oligomerization domain; or c) signal peptide, human oligomerization domain, display peptide, and transmembrane domain. In some embodiments, the recombinant fusion protein further comprises a cytosolic domain. In some embodiments, domains of the recombinant fusion protein are arranged from the N-terminus to the C-terminus in the following orders: a) signal peptide, display polypeptide, human oligomerization domain, transmembrane domain, and cytosolic domain; b) signal peptide, display polypeptide, transmembrane domain, human oligomerization domain, and cytosolic domain; or c) signal peptide, human oligomerization domain, display peptide, transmembrane domain, and cytosolic domain.

In some embodiments, the enveloped particle comprises an enveloped viral-like particle, an enveloped virus, or an extracellular vesicle. In some embodiments, the extracellular vesicle comprises an exosome. In some embodiments, the extracellular vesicle comprises an ectosome. In some embodiments, the display polypeptide targets a cell surface protein that is oligomerized on a target cell for agonistic function. In some embodiments, the display polypeptide targets a cell surface protein that is oligomerized on a target cell for antagonistic function. In some embodiments, the cell surface protein is dimerized, and the human oligomerization domain is a dimerization domain. In some embodiments, the cell surface protein is trimerized, and the human oligomerization domain is a trimerization domain. In some embodiments, the cell surface protein is trimerized, and the human oligomerization domain is a tetramerization domain.

In some embodiments, the display polypeptide comprises a type I transmembrane polypeptide, a type II transmembrane polypeptide, a type III transmembrane polypeptide, a GPI-anchored polypeptide, a secreted polypeptide, or a multi-pass transmembrane polypeptide. In some embodiments, the type I transmembrane polypeptide comprises ACE2, TIGIT, or LAG-3. In some embodiments, the type II transmembrane polypeptide comprises OX40-L, TNF-α, or APRIL. In some embodiments, the type III transmembrane polypeptide comprises TACI. In some embodiments, the GPI-anchored polypeptide comprises CD160 or CD48. In some embodiments, the secreted polypeptide comprises IFN-β or LT-α. In some embodiments, the multi-pass transmembrane polypeptide comprises CXCR-4. In some embodiments, the display polypeptide comprises an antibody, a receptor polypeptide, a cytokine, an immune checkpoint polypeptide, a chemokine, or an adhesion factor. In some embodiments, the receptor polypeptide comprises ACE2 or CXCR-4. In some embodiments, the cytokine comprises IFN-β or IFNγ. In some embodiments, the immune checkpoint polypeptide comprises PD-L1 or PD-1. In some embodiments, the chemokine comprises CXCL-12. In some embodiments, the adhesion factor comprises ICAM-1. In some embodiments, the display polypeptide comprises an amino acid sequence that has at least 85% sequence identity to an amino acid sequence according to SEQ ID NOs: 7-26. In some embodiments, the display polypeptide comprises an amino acid sequence that has at least 95% sequence identity to an amino acid sequence according to SEQ ID NOs: 7-26.

In some embodiments, the transmembrane domain anchors the recombinant fusion protein to a bilayer of the enveloped particle. In some embodiments, the transmembrane domain comprises the transmembrane domain of a Vesicular Stomatitis virus glycoprotein (VSV-G). In some embodiments, the recombinant fusion protein comprises the transmembrane domain and cytosolic domain of a Vesicular Stomatitis virus glycoprotein (VSV-G). In some embodiments, the transmembrane domain comprises the transmembrane domain of a Dengue E protein. In some embodiments, the recombinant fusion protein comprises the transmembrane domain and cytosolic domain of a Dengue E protein. In some embodiments, the transmembrane domain comprises the transmembrane domain of influenza Neuraminidase (NA). In some embodiments, the recombinant fusion protein comprises the transmembrane domain and cytosolic domain of influenza Neuraminidase (NA). In some embodiments, the transmembrane domain comprises an amino acid sequence that has at least 85% sequence identity to an amino acid sequence according to SEQ ID NOs: 27-35. In some embodiments, the transmembrane domain comprises an amino acid sequence that has at least 95% sequence identity to an amino acid sequence according to SEQ ID NOs: 27-35.

In some embodiments, when the recombinant fusion protein is expressed on the surface of the enveloped particle, the recombinant fusion protein is expressed at a valency of about 10 copies on the surface of the enveloped particle. In some embodiments, when the recombinant fusion protein is expressed on the surface of the enveloped particle, the recombinant fusion protein is expressed at a valency of about 10 to 15 copies on the surface of the enveloped particle. In some embodiments, when the recombinant fusion protein is expressed on the surface of the enveloped particle, the recombinant fusion protein is expressed at a valency of at least about 25 copies on the surface of the enveloped particle. In some embodiments, when the recombinant fusion protein is expressed on the surface of the enveloped particle, the recombinant fusion protein is expressed at a valency of at least about 50 copies on the surface of the enveloped particle. In some embodiments, when the recombinant fusion protein is expressed on the surface of the enveloped particle, the recombinant fusion protein is expressed at a valency of at least about 100 copies on the surface of the enveloped particle. In some embodiments, when the recombinant fusion protein is expressed on the surface of the enveloped particle, the recombinant fusion protein is expressed at a valency of at least about 200 copies on the surface of the enveloped particle. In some embodiments, when the recombinant fusion protein is expressed on the surface of the enveloped particle, the recombinant fusion protein is expressed at a valency of at least about 400 copies on the surface of the enveloped particle. In some embodiments, when the recombinant fusion protein is expressed on the surface of the enveloped particle, the recombinant fusion protein is expressed at a valency of at least about 600 copies on the surface of the enveloped particle. In some embodiments, when the recombinant fusion protein is expressed on the surface of the enveloped particle, the recombinant fusion protein is expressed at least about 1000 copies on the surface of the enveloped particle. In some embodiments, when the recombinant fusion protein is expressed on the surface of the enveloped particle, the recombinant fusion protein is expressed at least about 2000 copies on the surface of the enveloped particle.

Disclosed herein, in certain embodiments, are an enveloped particle comprising the recombinant fusion protein according to the embodiments described above. Disclosed herein, in certain embodiments, are a composition comprising a nucleic acid sequence that encodes the recombinant fusion protein according to the embodiments described above.

Disclosed herein, in certain embodiments, are an enveloped particle comprising: a) a first recombinant fusion protein that is displayed in an oligomeric format on the surface of the enveloped particle wherein the first recombinant fusion protein comprises a transmembrane domain, a display polypeptide, and a human oligomerization domain; and b) a second recombinant fusion protein that is displayed in a monomeric format on the surface of the enveloped particle wherein the second recombinant fusion protein comprises a transmembrane domain and a display polypeptide. In some embodiments, the human oligomerization domain comprises a human coiled coil motif. In some embodiments, the human oligomerization domain is a dimerization domain. In some embodiments, the human oligomerization domain comprises a human fibrinogen-like protein 1 coiled coil (hFcc) domain. In some embodiments, the human oligomerization domain is a trimerization domain. In some embodiments, the human oligomerization domain comprises a LINE-1 retrotransposon orf1p coiled coil (L1cc) domain. In some embodiments, the human oligomerization domain comprises a human lung surfactant protein D coiled coil (hLSPDcc) domain. In some embodiments, the human oligomerization domain comprises a human tetranectin coiled coil (hTETNcc) domain. In some embodiments, the human oligomerization domain is a tetramerization domain. In some embodiments, the human oligomerization domain comprises a human guanylate-binding protein 1 coiled coil (hGBP1cc) domain. In some embodiments, the human oligomerization domain comprises an amino acid sequence that has at least 85% sequence identity to an amino acid sequence according to SEQ ID NOs: 1-6. In some embodiments, the human oligomerization domain comprises an amino acid sequence that has at least 95% sequence identity to an amino acid sequence according to SEQ ID NOs: 1-6.

In some embodiments, when the first recombinant fusion protein is expressed on the surface of the enveloped particle, the human oligomerization domain is outside of the enveloped particle. In some embodiments, when the first recombinant fusion protein is expressed on the surface of the enveloped particle, the human oligomerization domain is outside of the enveloped particle and adjacent to a signal peptide. In some embodiments, when the first recombinant fusion protein is expressed on the surface of the enveloped particle, the human oligomerization domain is inside of the enveloped particle. In some embodiments, when the first recombinant fusion protein is expressed on the surface of the enveloped particle, the human oligomerization domain is inside of the enveloped particle and adjacent to the transmembrane domain.

In some embodiments, the first recombinant fusion protein further comprises a signal peptide. In some embodiments, domains of the first recombinant fusion protein are arranged from the N-terminus to the C-terminus in the following orders: a) signal peptide, display polypeptide, human oligomerization domain, and transmembrane domain; b) signal peptide, display polypeptide, transmembrane domain, and human oligomerization domain; or c) signal peptide, human oligomerization domain, display peptide, and transmembrane domain. In some embodiments, the first recombinant fusion protein further comprises a cytosolic domain. In some embodiments, domains of the first recombinant fusion protein are arranged from the N-terminus to the C-terminus in the following orders: a) signal peptide, display polypeptide, human oligomerization domain, transmembrane domain, and cytosolic domain; b) signal peptide, display polypeptide, transmembrane domain, human oligomerization domain, and cytosolic domain; or c) signal peptide, human oligomerization domain, display peptide, transmembrane domain, and cytosolic domain.

In some embodiments, the enveloped particle comprises an enveloped viral-like particle, an enveloped virus, an extracellular vesicle. In some embodiments, the extracellular vesicle comprises an exosome. In some embodiments, the extracellular vesicle comprises an ectosome. In some embodiments, the first recombinant fusion protein and the second recombinant fusion protein have the same display polypeptide. In some embodiments, the first recombinant fusion protein and the second recombinant fusion protein have different display polypeptides. In some embodiments, the display polypeptide targets a cell surface protein that is oligomerized on a target cell for agonistic function. In some embodiments, the display polypeptide targets a cell surface protein that is oligomerized on a target cell for antagonistic function. In some embodiments, the cell surface protein is dimerized and the human oligomerization domain is a dimerization domain. In some embodiments, the cell surface protein is trimerized and the human oligomerization domain is a trimerization domain. In some embodiments, the cell surface protein is tetramerized and the human oligomerization domain is a tetramerization domain.

In some embodiments, the display polypeptide comprises a type I transmembrane polypeptide, a type II transmembrane polypeptide, a type III transmembrane polypeptide, a GPI-anchored polypeptide, a secreted polypeptide, or a multi-pass transmembrane polypeptide. In some embodiments, the type I transmembrane polypeptide comprises ACE2, TIGIT, or LAG-3. In some embodiments, the type II transmembrane polypeptide comprises OX40-L, TNF-α, or APRIL. In some embodiments, the type III transmembrane polypeptide comprises TACI. In some embodiments, the GPI-anchored polypeptide comprises CD160 or CD48. In some embodiments, the secreted polypeptide comprises IFN-β or LT-α. In some embodiments, the multi-pass transmembrane polypeptide comprises CXCR-4. In some embodiments, the display polypeptide comprises an antibody, a receptor polypeptide, a cytokine, an immune checkpoint polypeptide, a chemokine, or an adhesion factor. In some embodiments, the receptor polypeptide comprises ACE2 or CXCR-4. In some embodiments, the cytokine comprises IFN-β or IFNγ. In embodiments, the chemokine comprises CXCL-12. In some embodiments, the adhesion factor comprises ICAM-1. In some embodiments, the display polypeptide comprises an amino acid sequence that has at least 85% sequence identity to an amino acid sequence according to SEQ ID NOs: 7-26. In some embodiments, the display polypeptide comprises an amino acid sequence that has at least 95% sequence identity to an amino acid sequence according to SEQ ID NOs: 7-26.

In some embodiments, the first recombinant fusion protein and the second recombinant fusion protein have the same transmembrane domains. In some embodiments, the first recombinant fusion protein and the second recombinant fusion protein have different transmembrane domains. In some embodiments, the transmembrane domain anchors the recombinant fusion protein to a bilayer of the enveloped particle. In some embodiments, the transmembrane domain comprises the transmembrane domain of a Vesicular Stomatitis virus glycoprotein (VSV-G). In some embodiments, the first recombinant fusion protein comprises the transmembrane domain and cytosolic domain of a Vesicular Stomatitis virus glycoprotein (VSV-G). In some embodiments, the transmembrane domain comprises the transmembrane domain of a Dengue E protein. In some embodiments, the first recombinant fusion protein comprises the transmembrane domain and cytosolic domain of a Dengue E protein. In some embodiments, the transmembrane domain comprises the transmembrane domain of influenza Neuraminidase (NA). In some embodiments, the first recombinant fusion protein comprises the transmembrane domain and cytosolic domain of influenza Neuraminidase (NA). In some embodiments, the transmembrane domain comprises an amino acid sequence that has at least 85% sequence identity to an amino acid sequence according to SEQ ID NOs: 27-35. In some embodiments, the transmembrane domain comprises an amino acid sequence that has at least 95% sequence identity to an amino acid sequence according to SEQ ID NOs: 27-35.

In some embodiments, when the first recombinant fusion protein is expressed on the surface of the enveloped particle the first recombinant fusion protein is expressed at a valency of about 10 copies on the surface of the enveloped particle. In some embodiments, when the first recombinant fusion protein is expressed on the surface of the enveloped particle the first recombinant fusion protein is expressed at a valency of about 10 to 15 copies on the surface of the enveloped particle. In some embodiments, when the first recombinant fusion protein is expressed on the surface of the enveloped particle the first recombinant fusion protein is expressed at a valency of at least about 25 copies on the surface of the enveloped particle. In some embodiments, when the first recombinant fusion protein is expressed on the surface of the enveloped particle the first recombinant fusion protein is expressed at a valency of at least about 50 copies on the surface of the enveloped particle. In some embodiments, when the first recombinant fusion protein is expressed on the surface of the enveloped particle the first recombinant fusion protein is expressed at a valency of at least about 100 copies on the surface of the enveloped particle. In some embodiments, when the first recombinant fusion protein is expressed on the surface of the enveloped particle the first recombinant fusion protein is expressed at a valency of at least about 200 copies on the surface of the enveloped particle. In some embodiments, when the first recombinant fusion protein is expressed on the surface of the enveloped particle the first recombinant fusion protein is expressed at a valency of at least about 400 copies on the surface of the enveloped particle. In some embodiments, when the first recombinant fusion protein is expressed on the surface of the enveloped particle the first recombinant fusion protein is expressed at a valency of at least about 600 copies on the surface of the enveloped particle. In some embodiments, when the first recombinant fusion protein is expressed on the surface of the enveloped particle the first recombinant fusion protein is expressed on the surface of the enveloped particle at a valency of at least about 1000 copies on the surface of the enveloped particle.

In some embodiments, when the second recombinant fusion protein is expressed on the surface of the enveloped particle the second recombinant fusion protein is expressed at a valency of about 10 copies on the surface of the enveloped particle. In some embodiments, when the second recombinant fusion protein is expressed on the surface of the enveloped particle the second recombinant fusion protein is expressed at a valency of about 10 to 15 copies on the surface of the enveloped particle. In some embodiments, wherein when the second recombinant fusion protein is expressed on the surface of the enveloped particle the second recombinant fusion protein is expressed at a valency of at least about 25 copies on the surface of the enveloped particle. In some embodiments, when the second recombinant fusion protein is expressed on the surface of the enveloped particle the second recombinant fusion protein is expressed at a valency of at least about 50 copies on the surface of the enveloped particle. In some embodiments, when the second recombinant fusion protein is expressed on the surface of the enveloped particle the second recombinant fusion protein is expressed at a valency of at least about 100 copies on the surface of the enveloped particle. In some embodiments, when the second recombinant fusion protein is expressed on the surface of the enveloped particle the second recombinant fusion protein is expressed at a valency of at least about 200 copies on the surface of the enveloped particle. In some embodiments, when the second recombinant fusion protein is expressed on the surface of the enveloped particle the second recombinant fusion protein is expressed at a valency of at least about 400 copies on the surface of the enveloped particle. In some embodiments, when the second recombinant fusion protein is expressed on the surface of the enveloped particle the second recombinant fusion protein is expressed at a valency of at least about 600 copies on the surface of the enveloped particle. In some embodiments, when the second recombinant fusion protein is expressed on the surface of the enveloped particle the second recombinant fusion protein is expressed on the surface of the enveloped particle at a valency of at least about 1000 copies on the surface of the enveloped particle. when the second recombinant fusion protein is expressed on the surface of the enveloped particle the second recombinant fusion protein is expressed on the surface of the enveloped particle at a valency of at least about 2000 copies on the surface of the enveloped particle.

Disclosed herein, in certain embodiments, are a composition comprising a nucleic acid sequence that encodes the recombinant fusion protein according to the embodiments described above. Disclosed herein, in certain embodiments, are a composition comprising a nucleic acid sequence that encodes the first recombinant fusion protein according to the embodiments described above; and the second recombinant fusion protein according to the embodiments described above. In some embodiments, the composition further comprises a second nucleic acid sequence that encodes one or more packaging viral proteins. In some embodiments, the one or more packaging viral proteins is a lentiviral protein, a retroviral protein, an adenoviral protein, or combinations thereof. In some embodiments, the one or more packaging viral proteins comprises gag, pol, pre, tat, rev, or combinations thereof. In some embodiments, the composition further comprises a third nucleic acid sequence that encodes a reporter, a therapeutic molecule, or combinations thereof. In some embodiments, the reporter is a fluorescent protein. In some embodiments, the reporter is a luciferase. In some embodiments, the fluorescent protein is green fluorescent protein. In some embodiments, the therapeutic molecule is an immune modulating protein, a cellular signal modulating molecule, a proliferation modulating molecule, a cell death modulating molecule, or combinations thereof.

In some embodiments, the nucleic acid sequence that encodes the recombinant fusion protein and the second nucleic acid sequence and the third nucleic acid sequence are within a same vector. In some embodiments, the nucleic acid sequence that encodes the first recombinant fusion protein and the second recombinant fusion protein and the second nucleic acid sequence and the third nucleic acid sequence are within a same vector. In some embodiments, the nucleic acid sequence that encodes the recombinant fusion protein and the second nucleic acid sequence and the third nucleic acid sequence are within different vectors. In some embodiments, the nucleic acid sequence that encodes the first recombinant fusion protein and the second recombinant fusion protein and the second nucleic acid sequence and the third nucleic acid sequence are within different vectors. In some embodiments, the vector is a lentivirus vector, an adenovirus vector, or an adeno-associated virus vector.

Disclosed herein, in certain embodiments, are a method of agonizing or antagonizing a cell that has an oligomerized cell surface protein comprising contacting the cell with an enveloped particle comprising a first recombinant fusion protein that is displayed in an oligomeric format on the surface of the enveloped particle, wherein the first recombinant fusion protein comprises a transmembrane domain, a display polypeptide, and a human oligomerization domain. In some embodiments, the enveloped particle further comprises a second recombinant fusion protein that is displayed in a monomeric format on the surface of the enveloped particle, wherein the second recombinant fusion protein comprises a transmembrane domain and a display polypeptide. In some embodiments, the human oligomerization domain comprises a human coiled coil motif. In some embodiments, the human oligomerization domain is a dimerization domain. In some embodiments, the human oligomerization domain comprises a human fibrinogen-like protein 1 coiled coil (hFcc) domain. In some embodiments, the human oligomerization domain is a trimerization domain. In some embodiments, the human oligomerization domain comprises a LINE-1 retrotransposon orf1p coiled coil (L1cc) domain. In some embodiments, the human oligomerization domain comprises a human lung surfactant protein D coiled coil (hLSPDcc) domain. In some embodiments, the human oligomerization domain comprises a human tetranectin coiled coil (hTETNcc) domain. In some embodiments, the human oligomerization domain is a tetramerization domain. In some embodiments, the human oligomerization domain comprises a human guanylate-binding protein 1 coiled coil (hGBP1cc) domain. In some embodiments, the human oligomerization domain comprises an amino acid sequence that has at least 85% sequence identity to an amino acid sequence according to SEQ ID NOs: 1-6. In some embodiments, the human oligomerization domain comprises an amino acid sequence that has at least 95% sequence identity to an amino acid sequence according to SEQ ID NOs: 1-6.

In some embodiments, when the first recombinant fusion protein is expressed on the surface of the enveloped particle, the human oligomerization domain is outside of the enveloped particle. In some embodiments, when the first recombinant fusion protein is expressed on the surface of the enveloped particle, the human oligomerization domain is outside of the enveloped particle and adjacent to a signal peptide. In some embodiments, when the first recombinant fusion protein is expressed on the surface of the enveloped particle, the human oligomerization domain is inside of the enveloped particle. In some embodiments, when the first recombinant fusion protein is expressed on the surface of the enveloped particle, the human oligomerization domain is inside of the enveloped particle and adjacent to the transmembrane domain.

In some embodiments, the first recombinant fusion protein comprises a signal peptide. In some embodiments, domains of the first recombinant fusion protein are arranged from the N-terminus to the C-terminus in the following orders: a) signal peptide, display polypeptide, human oligomerization domain, and transmembrane domain; b) signal peptide, display polypeptide, transmembrane domain, and human oligomerization domain; or c) signal peptide, human oligomerization domain, display peptide, and transmembrane domain. In some embodiments, the first recombinant fusion protein further comprises a cytosolic domain. In some embodiments, domains of the first recombinant fusion protein are arranged from the N-terminus to the C-terminus in the following orders: a) signal peptide, display polypeptide, human oligomerization domain, transmembrane domain, and cytosolic domain; b) signal peptide, display polypeptide, transmembrane domain, human oligomerization domain, and cytosolic domain; or c) signal peptide, human oligomerization domain, display peptide, transmembrane domain, and cytosolic domain.

In some embodiments, the enveloped particle comprises an enveloped viral-like particle, an enveloped virus, an extracellular vesicle. In some embodiments, the extracellular vesicle comprises an exosome. In some embodiments, the extracellular vesicle comprises an ectosome. In some embodiments, the first recombinant fusion protein and the second recombinant fusion protein have the same display polypeptide. In some embodiments, the first recombinant fusion protein and the second recombinant fusion protein have different display polypeptides. In some embodiments, the display polypeptide targets the cell surface protein that is oligomerized on a target cell for agonistic function. In some embodiments, the display polypeptide targets the cell surface protein that is oligomerized on a target cell for antagonistic function. In some embodiments, the cell surface protein is dimerized and the coiled coil oligomerization domain is a dimerization domain. In some embodiments, the cell surface protein is trimerized and the coiled coil oligomerization domain is a trimerization domain. In some embodiments, the cell surface protein is tetramerized and the coiled coil oligomerization domain is a tetramerization domain.

In some embodiments, the display polypeptide comprises a type I transmembrane polypeptide, a type II transmembrane polypeptide, a type III transmembrane polypeptide, a GPI-anchored polypeptide, a secreted polypeptide, or a multi-pass transmembrane polypeptide. In some embodiments, the type I transmembrane polypeptide comprises ACE2, TIGIT, or LAG-3. In some embodiments, the type II transmembrane polypeptide comprises OX40-L, TNF-α, or APRIL. In some embodiments, the type III transmembrane polypeptide comprises TACI. In some embodiments, the GPI-anchored polypeptide comprises CD160 or CD48. In some embodiments, the secreted polypeptide comprises IFN-β or LT-α. In some embodiments, the multi-pass transmembrane polypeptide comprises CXCR-4. In some embodiments, the display polypeptide comprises an antibody, a receptor polypeptide, a cytokine, an immune checkpoint polypeptide, a chemokine, or an adhesion factor. In some embodiments, the receptor polypeptide comprises ACE2 or CXCR-4. In some embodiments, the cytokine comprises IFN-β or IFNγ. In some embodiments, the immune checkpoint polypeptide comprises PD-L1 or PD-1. In some embodiments, the chemokine comprises CXCL-12. In some embodiments, the adhesion factor comprises ICAM-1. In some embodiments, the display polypeptide comprises an amino acid sequence that has at least 85% sequence identity to an amino acid sequence according to SEQ ID NOs: 7-26. In some embodiments, the display polypeptide comprises an amino acid sequence that has at least 95% sequence identity to an amino acid sequence according to SEQ ID NOs: 7-26.

In some embodiments, the first recombinant fusion protein and the second recombinant fusion protein have the same transmembrane domains. In some embodiments, the first recombinant fusion protein and the second recombinant fusion protein have different transmembrane domains. In some embodiments, the transmembrane domain anchors the recombinant fusion protein to a bilayer of the enveloped particle. In some embodiments, the transmembrane domain comprises the transmembrane domain of a Vesicular Stomatitis virus glycoprotein (VSV-G). In some embodiments, the transmembrane domain comprises the transmembrane domain and cytosolic domain of a Vesicular Stomatitis virus glycoprotein (VSV-G). In some embodiments, the transmembrane domain comprises the transmembrane domain of a Dengue E protein. In some embodiments, the transmembrane domain comprises the transmembrane domain and cytosolic domain of a Dengue E protein. In some embodiments, the transmembrane domain comprises the transmembrane domain of influenza Neuraminidase (NA). In some embodiments, the transmembrane domain comprises the transmembrane domain and cytosolic domain of influenza Neuraminidase (NA). In some embodiments, the transmembrane domain comprises an amino acid sequence that has at least 85% sequence identity to an amino acid sequence according to SEQ ID NOs: 27-35. In some embodiments, the transmembrane domain comprises an amino acid sequence that has at least 95% sequence identity to an amino acid sequence according to SEQ ID NOs: 27-35.

Disclosed herein, in certain embodiments, is a multivalent particle (MVP) comprising an enveloped particle, wherein the MVP displays at least about 10 copies of a peptide on a surface of the MVP, wherein at least one surface protein on the surface of the MVP comprises an oligomerized domain, wherein the peptide forms multivalent interactions with a cognate ligand on a target cell or a target virus.

Disclosed herein, in certain embodiments, is a method of using a multivalent particle (MVP), wherein the MVP displays an oligomerized peptide as an immunogen to generate protective immunity against a target antigen, wherein the target antigen is a bacterial antigen, a viral antigen, a fungal antigen, or a tumor antigens, wherein at least about 10 copies of the oligomerized peptide on a surface of the MVP, wherein at least one surface protein on the MVP comprises an oligomerized domain.

Disclosed herein, in certain embodiments, is a method of using a multivalent particle (MVP), wherein the MVP displays an oligomerized peptide as an immunogen to generate protective immunity against a target antigen, wherein the target antigen is a bacterial antigen, a viral antigen, a fungal antigen, or a tumor antigen, wherein the MVP comprises a double stranded ribonucleic viral genome as a toll-like receptor agonist adjuvant.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A shows a schematic of an example oligomeric peptide display vector, where SP=signal peptide; OD=oligomerization domain (e.g. VSV-G D4, LINE1 coiled-coil trimerization domain); TM=VSV-G transmembrane domain; and C=VSV-G cytosolic tail domain. FIG. 1B provides a schematic of the VSV-G protein transcriptome and a ribbon diagram of the VSV-G protein.

FIG. 2 illustrates a parallel, left-handed homodimer coiled coil protein (left), and a coiled coil hexamer (right).

FIG. 3 illustrate different organization motifs of oligomerization domains of the disclosure.

FIG. 4 illustrates the structure of the LINE-1 retrotransposon ORF1p oligomerization domain. PANEL A illustrates the oligomerization domain having a L1cc trimerization domain next to an RNA recognition motif (RRM), followed by a C-terminal domain (CTD). PANEL B illustrates the protein structure of the L1cc monomer coiled coil, RRM, and CTD. PANEL C illustrates the protein structure of the L1cc trimer coiled coil, RRM, and CTD.

FIG. 5 illustrates an example design of multivalent particle (MVP) display constructs utilizing the human Line1 coiled-coil (L1cc) trimerization domain, which includes a signal peptide, a display peptide (Peptide), and a humanized oligomeric display carrier comprising L1cc coiled coil, transmembrane domain, and a C-terminal.

FIG. 6A is a schematic that illustrates the production of viral-like particle (VLP) MVPs containing RNA genomes that display L1cc fusion peptides. FIG. 6B is a schematic that illustrates the production of VLP MVPs without genomes. FIG. 6C is a schematic that illustrates the production of extracellular vesicle (EV) MVPs displaying L1cc fusion peptides.

FIG. 7A depicts a quantitative western blot analysis of the copy numbers of displayed humanized L1cc fusion peptides on MVPs. FIG. 7B depicts a non-reducing page analysis of oligomerization patterns of displayed L1cc fusion peptides and D4 fusion peptides.

FIG. 8A illustrates configurations of ACE2 L1cc display peptide oligomerization domains, with a L1cc domain preceding (1) or following (2) a VSV-G transmembrane domain, and FIG. 8B depicts a quantitative western blot analysis of ACE2 copy number in decoy MVPs displaying either of the display construct variations.

FIG. 9 is a chart that illustrates the percent neutralization of SARS COV-2 spike variants by decoy MVPs displaying ACE2-L1cc.

FIG. 10 is a chart that illustrates the percent neutralization of E484Q pseudovirus by the ACE2 decoy MVPs derived from both configurations of L1cc display vectors depicted in FIG. 8A.

FIG. 11A-11B show the effect of administering humanized decoy MVPs displaying ACE2-L1cc to hACE2 transgenic mice with lethal SARS COV-2 infections on survival (%) and % body weight change.

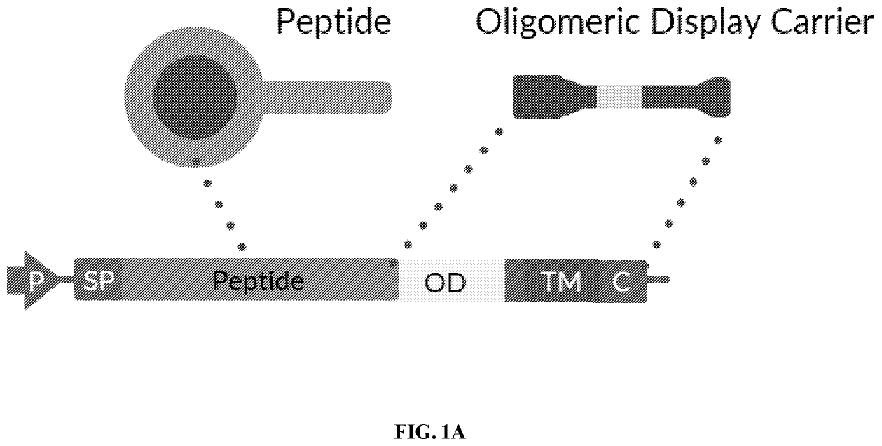

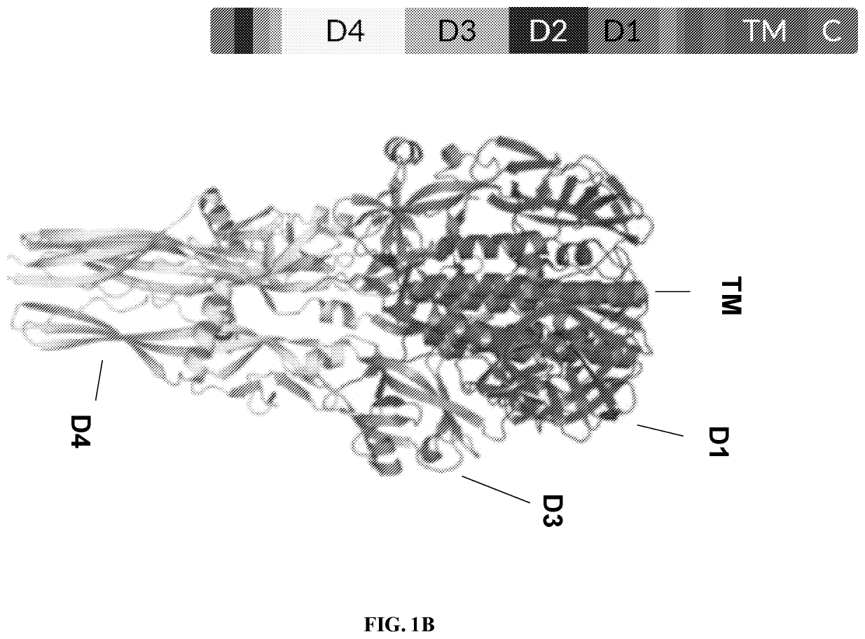

### DETAILED DESCRIPTION OF THE INVENTION

Multivalent interactions play critical roles in a variety of biological processes. A multivalent ligand can bind to one or more multiple receptors with enhanced functional affinity. Such interactions can induce receptor clustering and lead to signal transduction. However, despite the beneficial role of multivalent interactions in normal physiological processes, multivalent interactions also underlie cell regulation that contributes to disease pathogenesis. Multivalence can exponentially enhance the functional affinity of ligand-receptor interactions and may create significant barriers for small molecule or antibody drugs.

To overcome such challenges associated with targeting disease pathogenesis characterized by multivalence, described herein are recombinant fusion proteins that comprise coiled coil oligomerization domains that when expressed on the surface of enveloped particle are displayed in an oligomerized format. In some embodiments, the recombinant fusion protein is expressed on the surface of the enveloped particle at a valency of 10 or more copies. Without being bound by a particular theory, expression of the recombinant fusion proteins at a high valency on the enveloped particle can effectively mimic the multivalent target involved in the disease progression and counteract the multivalent target through antagonism or agonism. The multivalent target can be further mimicked through the use of a coiled coil oligomerization domain in combination with a high copy number to counteract the multivalent target more effectively than administration of a single agent such as isolated antibodies or small molecules.

### Recombinant Fusion Proteins

Coiled coil domains constitute a series of structurally repetitive motifs that contain a coiled group of identical 2 to 7 α-helices (FIG. 2). Coiled coil domains are found in numerous human proteins, and can induce a range of oligomerization states from dimers to heptamers. Coil domains from a variety of human proteins can be used to create various oligomeric patterns of peptide display on MVPs.

The MVP display constructs described herein can enable multivalent display of fusion peptides on the surface of VLPs and EVs, in varied oligomeric configuration and at copy numbers comparable to or greater than their viral display counterparts. Since these coiled coil oligomerization peptides are originated from human proteins, the multivalent therapeutics produced by using these MVP display constructs can display diminished immunogenicity.

Disclosed herein are recombinant fusion proteins comprising a transmembrane domain, a display polypeptide, and a coiled coil oligomerization domain wherein when the recombinant fusion protein is expressed on the surface of an enveloped particle, the recombinant fusion protein is displayed in an oligomeric format.

Disclosed herein, in certain embodiments, are a recombinant fusion protein comprising a transmembrane domain, a display polypeptide, and a human oligomerization domain, wherein when the recombinant fusion protein is expressed on a surface of an enveloped particle, the recombinant fusion protein is displayed in an oligomeric format. In some embodiments, the human oligomerization domain comprises a coiled coil motif. In some embodiments, the human oligomerization domain is a dimerization domain. In some embodiments, the human oligomerization domain comprises a human fibrinogen-like protein 1 coiled coil (hFcc) domain. In some embodiments, the human oligomerization domain is a trimerization domain. In some embodiments, the human oligomerization domain comprises a LINE-1 retrotransposon orf1p coiled coil (L1cc) domain. In some embodiments, the human oligomerization domain comprises a human lung surfactant protein D coiled coil (hLSPDcc) domain. In some embodiments, the human oligomerization domain comprises a human tetranectin coiled coil (hTETNcc) domain. In some embodiments, the human oligomerization domain is a tetramerization domain. In some embodiments, the human oligomerization domain comprises a human guanylate-binding protein 1 coiled coil (hGBP1cc) domain.

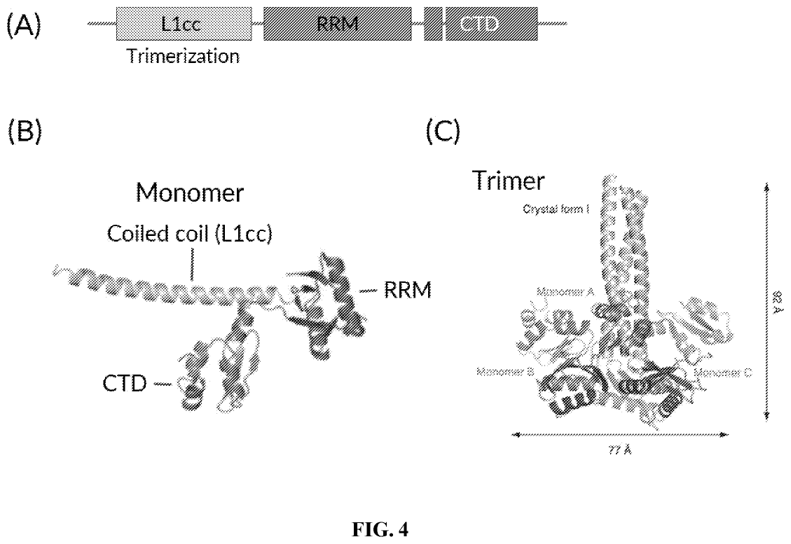

LINE-1 (L1) retrotransposon (FIG. 2), which accounts for roughly seventeen percent of the human genome, contains two open reading frames (orf), the first of which encodes a highly unique, 500 amino acid protein (orf1p) including a LINE-1 retrotransposon orf1p coiled coil (L1cc) trimerization domain. L1cc can serve as a non-immunogenic domain for trimeric peptide display on MVPs in part due to the prevalence of the L1 retrotransposon in the human genome. FIG. 4 illustrates the structure of the LINE-1 retrotransposon ORF1p oligomerization domain. PANEL A illustrates the oligomerization domain having a L1cc trimerization domain next to an RNA recognition motif (RRM), followed by a C-terminal domain (CTD). PANEL B illustrates the protein structure of the L1cc monomer coiled coil, RRM, and CTD. PANEL C illustrates the protein structure of the L1cc trimer coiled coil, RRM, and CTD.

Other MVP display constructs can be designed to display fusion peptides in dimeric, trimeric, and tetrameric configurations, using coiled coil oligomerization domains from variety of human proteins, such as, for example, the domains provided in Table 1A. Table 1B shows the sequences of the exemplary oligomerization domains.

TABLE 1A
Exemplary Oligomerization Domains

| | | Coiled | Oligomerization |
|---|---|---|---|
| Domain | Origin | coil | Degree |
| L1cc | LINE-1 Retrotransposon orf1p | Yes | Trimer |
| | coiled coil | | |
| CXV | Human Collagen XV | No | Trimer |
| hLSPDcc | Human lung surfactant protein | Yes | Trimer |
| | D coiled coil | | |
| hTETNcc | Human tetranectin coiled coil | Yes | Trimer |
| hGBP1cc | Human guanylate-binding | Yes | Tetramer |
| | protein 1 coiled coil | | |
| hFcc | Human fibrinogen-like protein 1 | Yes | Dimer |
| | coiled coil | | |

TABLE 1B
Sequences of Exemplary Oligomerization Domains

| | | SEQ |
|---|---|---|
| | | ID |
| Domain | Sequence | NO. |
| L1cc | RSNYSELREDIQTKGKEVENFEKNLEECITRITN | 1 |
| | TEKCLKELMELKTKARELREECRSLRSRCDQLEE | |
| | RVSAMEDEMNEMKREGKFREKRIKRNEQSLQEIW | |
| | DYV | |
| CXV | NLVTAFSNMDDMLQKAHLVIEGTFIYLRDSTEFF | 2 |
| | IRVRDGWKKLQLGELIPIPA | |
| hLSPDcc | VASLRQQVEALQGQVQHLQAAFSQYKKVELFPN | 3 |
| hTETNcc | LKSRLDTLAQEVALLKEQQALQTVCLKGTKV | 4 |
| hGBP1cc | DQTLTEKEKEIEVERVKAESAQASAKMLHEMQRK | 5 |
| | NEQMMEQKERSYQEHLKQLTEKMENDRVQLLKEQ | |
| | ERTLALKLQEQEQL | |
| hFcc | AKVFSFILVTTALTMGREISALEDCAQEQMRLRA | 6 |
| | QVRLLETRVKQQQVKIKQLLQENEVQFLDKG | |

In some embodiments, the human oligomerization domain comprises an amino acid sequence that has at least 85% sequence identity to an amino acid sequence according to SEQ ID NOs: 1-6. In some embodiments, the human oligomerization domain comprises an amino acid sequence that has at least 95% sequence identity to an amino acid sequence according to SEQ ID NOs: 1-6.

In some embodiments, the coiled coil oligomerization domain comprises an amino acid sequence of at least 75% sequence identity to an amino acid sequence according to SEQ ID NO: 1. In some embodiments, the coiled coil oligomerization domain comprises an amino acid sequence of at least 80% sequence identity to an amino acid sequence according to SEQ ID NO: 1. In some embodiments, the coiled coil oligomerization domain comprises an amino acid sequence of at least 85% sequence identity to an amino acid sequence according to SEQ ID NO: 1. In some embodiments, the coiled coil oligomerization domain comprises an amino acid sequence of at least 90% sequence identity to an amino acid sequence according to SEQ ID NO: 1. In some embodiments, the coiled coil oligomerization domain comprises an amino acid sequence of at least 95% sequence identity to an amino acid sequence according to SEQ ID NO: 1. In some embodiments, the coiled coil oligomerization domain comprises an amino acid sequence of at least 97% sequence identity to an amino acid sequence according to SEQ ID NO: 1. In some embodiments, the coiled coil oligomerization domain comprises an amino acid sequence of at least 98% sequence identity to an amino acid sequence according to SEQ ID NO: 1. In some embodiments, the coiled coil oligomerization domain comprises an amino acid sequence of at least 99% sequence identity to an amino acid sequence according to SEQ ID NO: 1. In some embodiments, the coiled coil oligomerization domain comprises an amino acid sequence according to SEQ ID NO: 1.

In some embodiments, the coiled coil oligomerization domain comprises an amino acid sequence of at least 75% sequence identity to an amino acid sequence according to SEQ ID NO: 2. In some embodiments, the coiled coil oligomerization domain comprises an amino acid sequence of at least 80% sequence identity to an amino acid sequence according to SEQ ID NO: 2. In some embodiments, the coiled coil oligomerization domain comprises an amino acid sequence of at least 85% sequence identity to an amino acid sequence according to SEQ ID NO: 2. In some embodiments, the coiled coil oligomerization domain comprises an amino acid sequence of at least 90% sequence identity to an amino acid sequence according to SEQ ID NO: 2. In some embodiments, the coiled coil oligomerization domain comprises an amino acid sequence of at least 95% sequence identity to an amino acid sequence according to SEQ ID NO: 2. In some embodiments, the coiled coil oligomerization domain comprises an amino acid sequence of at least 97% sequence identity to an amino acid sequence according to SEQ ID NO: 2. In some embodiments, the coiled coil oligomerization domain comprises an amino acid sequence of at least 98% sequence identity to an amino acid sequence according to SEQ ID NO: 2. In some embodiments, the coiled coil oligomerization domain comprises an amino acid sequence of at least 99% sequence identity to an amino acid sequence according to SEQ ID NO: 2. In some embodiments, the coiled coil oligomerization domain comprises an amino acid sequence according to SEQ ID NO: 2.

In some embodiments, the coiled coil oligomerization domain comprises an amino acid sequence of at least 75% sequence identity to an amino acid sequence according to SEQ ID NO: 3. In some embodiments, the coiled coil oligomerization domain comprises an amino acid sequence of at least 80% sequence identity to an amino acid sequence according to SEQ ID NO: 3. In some embodiments, the coiled coil oligomerization domain comprises an amino acid sequence of at least 85% sequence identity to an amino acid sequence according to SEQ ID NO: 3. In some embodiments, the coiled coil oligomerization domain comprises an amino acid sequence of at least 90% sequence identity to an amino acid sequence according to SEQ ID NO: 3. In some embodiments, the coiled coil oligomerization domain comprises an amino acid sequence of at least 95% sequence identity to an amino acid sequence according to SEQ ID NO: 3. In some embodiments, the coiled coil oligomerization domain comprises an amino acid sequence of at least 97% sequence identity to an amino acid sequence according to SEQ ID NO: 3. In some embodiments, the coiled coil oligomerization domain comprises an amino acid sequence of at least 98% sequence identity to an amino acid sequence according to SEQ ID NO: 3. In some embodiments, the coiled coil oligomerization domain comprises an amino acid sequence of at least 99% sequence identity to an amino acid sequence according to SEQ ID NO: 3. In some embodiments, the coiled coil oligomerization domain comprises an amino acid sequence according to SEQ ID NO: 3.

In some embodiments, the coiled coil oligomerization domain comprises an amino acid sequence of at least 75% sequence identity to an amino acid sequence according to SEQ ID NO: 4. In some embodiments, the coiled coil oligomerization domain comprises an amino acid sequence of at least 80% sequence identity to an amino acid sequence according to SEQ ID NO: 4. In some embodiments, the coiled coil oligomerization domain comprises an amino acid sequence of at least 85% sequence identity to an amino acid sequence according to SEQ ID NO: 4. In some embodiments, the coiled coil oligomerization domain comprises an amino acid sequence of at least 90% sequence identity to an amino acid sequence according to SEQ ID NO: 4. In some embodiments, the coiled coil oligomerization domain comprises an amino acid sequence of at least 95% sequence identity to an amino acid sequence according to SEQ ID NO: 4. In some embodiments, the coiled coil oligomerization domain comprises an amino acid sequence of at least 97% sequence identity to an amino acid sequence according to SEQ ID NO: 4. In some embodiments, the coiled coil oligomerization domain comprises an amino acid sequence of at least 98% sequence identity to an amino acid sequence according to SEQ ID NO: 4. In some embodiments, the coiled coil oligomerization domain comprises an amino acid sequence of at least 99% sequence identity to an amino acid sequence according to SEQ ID NO: 4. In some embodiments, the coiled coil oligomerization domain comprises an amino acid sequence according to SEQ ID NO: 4.

In some embodiments, the coiled coil oligomerization domain comprises an amino acid sequence of at least 75% sequence identity to an amino acid sequence according to SEQ ID NO: 5. In some embodiments, the coiled coil oligomerization domain comprises an amino acid sequence of at least 80% sequence identity to an amino acid sequence according to SEQ ID NO: 5. In some embodiments, the coiled coil oligomerization domain comprises an amino acid sequence of at least 85% sequence identity to an amino acid sequence according to SEQ ID NO: 5. In some embodiments, the coiled coil oligomerization domain comprises an amino acid sequence of at least 90% sequence identity to an amino acid sequence according to SEQ ID NO: 5. In some embodiments, the coiled coil oligomerization domain comprises an amino acid sequence of at least 95% sequence identity to an amino acid sequence according to SEQ ID NO: 5. In some embodiments, the coiled coil oligomerization domain comprises an amino acid sequence of at least 97% sequence identity to an amino acid sequence according to SEQ ID NO: 5. In some embodiments, the coiled coil oligomerization domain comprises an amino acid sequence of at least 98% sequence identity to an amino acid sequence according to SEQ ID NO: 5. In some embodiments, the coiled coil oligomerization domain comprises an amino acid sequence of at least 99% sequence identity to an amino acid sequence according to SEQ ID NO: 5. In some embodiments, the coiled coil oligomerization domain comprises an amino acid sequence according to SEQ ID NO: 5.

In some embodiments, the coiled coil oligomerization domain comprises an amino acid sequence of at least 75% sequence identity to an amino acid sequence according to SEQ ID NO: 6. In some embodiments, the coiled coil oligomerization domain comprises an amino acid sequence of at least 80% sequence identity to an amino acid sequence according to SEQ ID NO: 6. In some embodiments, the coiled coil oligomerization domain comprises an amino acid sequence of at least 85% sequence identity to an amino acid sequence according to SEQ ID NO: 6. In some embodiments, the coiled coil oligomerization domain comprises an amino acid sequence of at least 90% sequence identity to an amino acid sequence according to SEQ ID NO: 6. In some embodiments, the coiled coil oligomerization domain comprises an amino acid sequence of at least 95% sequence identity to an amino acid sequence according to SEQ ID NO: 6. In some embodiments, the coiled coil oligomerization domain comprises an amino acid sequence of at least 97% sequence identity to an amino acid sequence according to SEQ ID NO: 6. In some embodiments, the coiled coil oligomerization domain comprises an amino acid sequence of at least 98% sequence identity to an amino acid sequence according to SEQ ID NO: 6. In some embodiments, the coiled coil oligomerization domain comprises an amino acid sequence of at least 99% sequence identity to an amino acid sequence according to SEQ ID NO: 6. In some embodiments, the coiled coil oligomerization domain comprises an amino acid sequence according to SEQ ID NO: 6.

In some instances, the oligomerization domain comprises an amino acid sequence comprising at least a portion having at least or about 10, 20, 30, 40, 50, 60, 70, 80, 90, 100, 110, 120, 130 amino acid sequences of any sequence according to Table 1B.

In some embodiments, when the recombinant fusion protein is expressed on the surface of the enveloped particle, the human oligomerization domain is outside of the enveloped particle. In some embodiments, when the recombinant fusion protein is expressed on the surface of the enveloped particle, the human oligomerization domain is outside of the enveloped particle and adjacent to a signal peptide. In some embodiments, when the recombinant fusion protein is expressed on the surface of the enveloped particle, the human oligomerization domain is inside of the enveloped particle. In some embodiments, when the recombinant fusion protein is expressed on the surface of the enveloped particle, and wherein the human oligomerization domain is inside of the enveloped particle and adjacent to the transmembrane domain.

In some embodiments, the recombinant fusion protein further comprises a signal peptide. In some embodiments, domains of the recombinant fusion protein are arranged from the N-terminus to the C-terminus in the following orders: a) signal peptide, display polypeptide, human oligomerization domain, and transmembrane domain; b) signal peptide, display polypeptide, transmembrane domain, and human oligomerization domain; or c) signal peptide, human oligomerization domain, display peptide, and transmembrane domain. In some embodiments, the recombinant fusion protein further comprises a cytosolic domain. In some embodiments, domains of the recombinant fusion protein are arranged from the N-terminus to the C-terminus in the following orders: a) signal peptide, display polypeptide, human oligomerization domain, transmembrane domain, and cytosolic domain; b) signal peptide, display polypeptide, transmembrane domain, human oligomerization domain, and cytosolic domain; or c) signal peptide, human oligomerization domain, display peptide, transmembrane domain, and cytosolic domain.

In some embodiments, the enveloped particle comprises an enveloped viral-like particle, an enveloped virus, or an extracellular vesicle. In some embodiments, the extracellular vesicle comprises an exosome. In some embodiments, the extracellular vesicle comprises an ectosome.

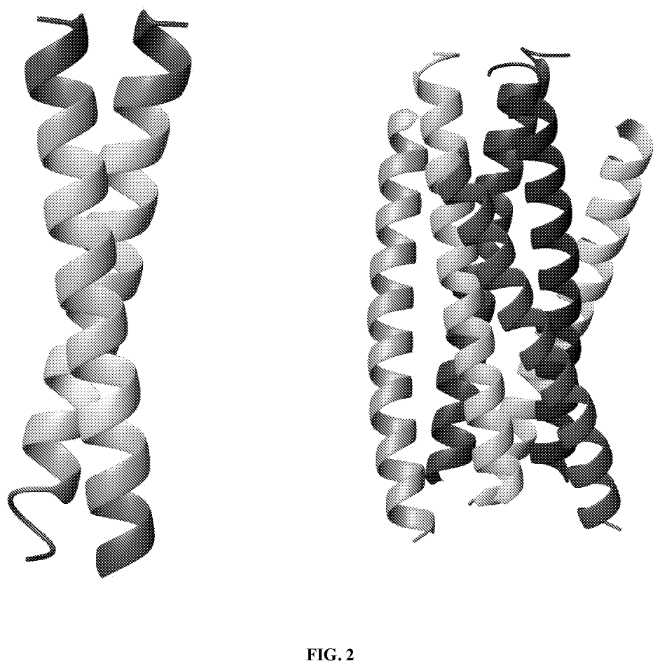

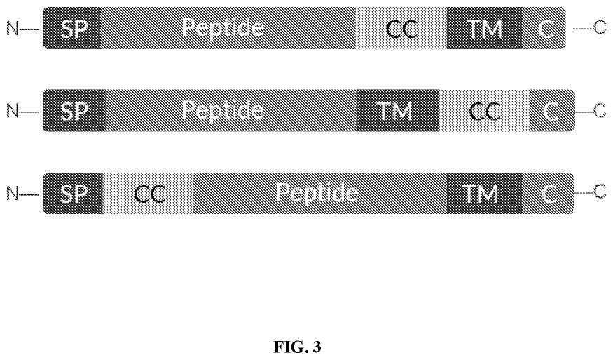

MVPs can be genetically programmed to display proteins in various configurations by modifying the display vector (FIG. 3). The coiled coil coiled coil oligomerization domain can be placed at various positions of the fusion peptide: (1) extracellular and juxtaposed to the transmembrane domain; (2) intracellular and juxtaposed to the transmembrane domain; (3) extracellular and after the signal peptide. Furthermore, various coiled coil oligomerization domains can be used for distinct surface display patterns that are suitable for the function of immune checkpoint molecules (TABLE 1). In addition to the L1cc trimerization domain, the coiled coil domains from human collagen XV, lung surfactant protein D, tetranectin, mannose binding protein, Factor X, and Fibrinogen can also be used to create oligomerized display patterns on the surface of viral-like particles (VLPs) or extracellular vesicles (EVs). With these display configurations, combinatorial MVPs can be programed with mixed monomeric, dimeric, trimeric, and tetrameric display patterns optimized to their function in target cell regulation or virus neutralization.

The display methods described above can be broadly used to generate patterned display of diverse classes of proteins with distinct structural features on VLPs and EVs.

Disclosed herein, in certain embodiments, are a composition comprising a nucleic acid sequence that encodes the recombinant fusion protein according to the embodiments described above. Disclosed herein, in certain embodiments, are a composition comprising a nucleic acid sequence that encodes the first recombinant fusion protein according to the embodiments described above; and the second recombinant fusion protein according to the embodiments described above. In some embodiments, the composition further comprises a second nucleic acid sequence that encodes one or more packaging viral proteins. In some embodiments, the one or more packaging viral proteins is a lentiviral protein, a retroviral protein, an adenoviral protein, or combinations thereof. In some embodiments, the one or more packaging viral proteins comprises gag, pol, pre, tat, rev, or combinations thereof. In some embodiments, the composition further comprises a third nucleic acid sequence that encodes a reporter, a therapeutic molecule, or combinations thereof. In some embodiments, the reporter is a fluorescent protein. In some embodiments, the reporter is a luciferase. In some embodiments, the fluorescent protein is green fluorescent protein. In some embodiments, the therapeutic molecule is an immune modulating protein, a cellular signal modulating molecule, a proliferation modulating molecule, a cell death modulating molecule, or combinations thereof.

In some embodiments, the nucleic acid sequence that encodes the recombinant fusion protein and the second nucleic acid sequence and the third nucleic acid sequence are within a same vector. In some embodiments, the nucleic acid sequence that encodes the first recombinant fusion protein and the second recombinant fusion protein and the second nucleic acid sequence and the third nucleic acid sequence are within a same vector. In some embodiments, the nucleic acid sequence that encodes the recombinant fusion protein and the second nucleic acid sequence and the third nucleic acid sequence are within different vectors. In some embodiments, the nucleic acid sequence that encodes the first recombinant fusion protein and the second recombinant fusion protein and the second nucleic acid sequence and the third nucleic acid sequence are within different vectors. In some embodiments, the vector is a lentivirus vector, an adenovirus vector, or an adeno-associated virus vector.

### Display Peptides

In some embodiments, the display polypeptide targets a cell surface protein that is oligomerized on a target cell for agonistic function. In some embodiments, the display polypeptide targets a cell surface protein that is oligomerized on a target cell for antagonistic function. In some embodiments, the cell surface protein is dimerized, and the human oligomerization domain is a dimerization domain. In some embodiments, the cell surface protein is trimerized, and the human oligomerization domain is a trimerization domain. In some embodiments, the cell surface protein is trimerized, and the human oligomerization domain is a tetramerization domain.

In some embodiments, the display polypeptide comprises a type I transmembrane polypeptide, a type II transmembrane polypeptide, a type III transmembrane polypeptide, a GPI-anchored polypeptide, a secreted polypeptide, or a multi-pass transmembrane polypeptide. In some embodiments, the type I transmembrane polypeptide comprises ACE2, TIGIT, or LAG-3. In some embodiments, the type II transmembrane polypeptide comprises OX40-L, TNF-α, or APRIL. In some embodiments, the type III transmembrane polypeptide comprises TACI. In some embodiments, the GPI-anchored polypeptide comprises CD160 or CD48. In some embodiments, the secreted polypeptide comprises IFN-β or LT-α. In some embodiments, the multi-pass transmembrane polypeptide comprises CXCR-4. In some embodiments, the display polypeptide comprises an antibody, a receptor polypeptide, a cytokine, an immune checkpoint polypeptide, a chemokine, or an adhesion factor. In some embodiments, the receptor polypeptide comprises ACE2 or CXCR-4. In some embodiments, the cytokine comprises IFN-β or IFNγ. In embodiments, the chemokine comprises CXCL-12. In some embodiments, the adhesion factor comprises ICAM-1.

TABLE 2
Exemplary Display Polypeptides

| Display | | | | | SEQ |
|---|---|---|---|---|---|
| Poly- | Full gene | Classi- | NCBI | | ID |
| peptide | name | fication | Ref No. | Amino Acid Sequence (N to C) | NO: |
| ACE2 | Angiotensin | type I | NC_000023.11 | MSSSSWLLLSLVAVTAAQSTIEEQAKTFLDKFNHEAEDL | 7 |
| | converting | trans- | | FYQSSLASWNYNTNITEENVQNMNNAGDKWSAFLKEQST | |
| | enzyme 2 | membrane | | LAQMYPLQEIQNLTVKLQLQALQQNGSSVLSEDKSKRLN | |
| | | poly- | | TILNTMSTIYSTGKVCNPDNPQECLLLEPGLNEIMANSL | |
| | | peptide; | | DYNERLWAWESWRSEVGKQLRPLYEEYVVLKNEMARANH | |
| | | receptor | | YEDYGDYWRGDYEVNGVDGYDYSRGQLIEDVEHTFEEIK | |
| | | | | PLYEHLHAYVRAKLMNAYPSYISPIGCLPAHLLGDMWGR | |
| | | | | FWTNLYSLTVPFGQKPNIDVTDAMVDQAWDAQRIFKEAE | |
| | | | | KFFVSVGLPNMTQGFWENSMLTDPGNVQKAVCHPTAWDL | |
| | | | | GKGDFRILMCTKVTMDDFLTAHAEMGAIQYDMAYAAQPF | |
| | | | | LLRNGANEGFHEAVGEIMSLSAATPKHLKSIGLLSPDFQ | |
| | | | | EDNETEINFLLKQALTIVGTLPFTYMLEKWRWMVFKGEI | |
| | | | | PKDQWMKKWWEMKREIVGVVEPVPHDETYCDPASLFHVS | |
| | | | | NDYSFIRYYTRTLYQFQFQEALCQAAKHEGPLHKCDISN | |
| | | | | STEAGQKLFNMLRLGKSEPWTLALENVVGAKNMNVRPLL | |
| | | | | NYFEPLFTWLKDQNKNSFVGWSTDWSPYADQSIKVRISL | |
| | | | | KSALGDKAYEWNDNEMYLFRSSVAYAMRQYFLKVKNQMI | |
| | | | | LFGEEDVRVANLKPRISFNFFVTAPKNVSDIIPRTEVEK | |
| | | | | AIRMSRSRINDAFRLNDNSLEFLGIQPTLGPPNQPPVS | |
| Human | T-cell | type I | NP_776160.2 | MRWCLLLIWAQGLRQAPLASGMMTGTIETTGNISAEKGG | 8 |
| TIGIT | imuno- | trans- | | SIILQCHLSSTTAQVTQVNWEQQDQLLAICNADLGWHIS | |
| | receptor | membrane | | PSFKDRVAPGPGLGLTLQSLTVNDTGEYFCIYHTYPDGT | |
| | with Ig and | poly- | | YTGRIFLEVLESSVAEHGARFQIPLLGAMAATLVVICTA | |
| | ITIM | peptide | | VIVVVALTRKKKALRIHSVEGDLRRKSAGQEEWSPSAPS | |
| | domains | | | PPGSCVQAEAAPAGLCGEQRGEDCAELHDYFNVLSYRSL | |
| | | | | GNCSFFTETG | |
| Human | Lymphocyte | type I | NP_002277.4 | MWEAQFLGLLFLQPLWVAPVKPLQPGAEVPVVWAQEGAP | 9 |
| LAG-3 | activation | trans- | | AQLPCSPTIPLQDLSLLRRAGVTWQHQPDSGPPAAAPGH | |
| | gene 3 | membrane | | PLAPGPHPAAPSSWGPRPRRYTVLSVGPGGLRSGRLPLQ | |
| | protein | poly- | | PRVQLDERGRQRGDFSLWLRPARRADAGEYRAAVHLRDR | |
| | | peptide | | ALSCRLRLRLGQASMTASPPGSLRASDWVILNCSFSRPD | |
| | | | | RPASVHWFRNRGQGRVPVRESPHHHLAESFLFLPQVSPM | |
| | | | | DSGPWGCILTYRDGFNVSIMYNLTVLGLEPPTPLTVYAG | |
| | | | | AGSRVGLPCRLPAGVGTRSFLTAKWTPPGGGPDLLVTGD | |
| | | | | NGDFTLRLEDVSQAQAGTYTCHIHLQEQQLNATVTLAII | |
| | | | | TVTPKSFGSPGSLGKLLCEVTPVSGQERFVWSSLDTPSQ | |
| | | | | RSFSGPWLEAQEAQLLSQPWQCQLYQGERLLGAAVYFTE | |
| | | | | LSSPGAQRSGRAPGALPAGHLLLFLILGVLSLLLLVTGA | |
| | | | | FGFHLWRRQWRPRRFSALEQGIHPPQAQSKIEELEQEPE | |
| | | | | PEPEPEPEPEPEPEPEQL | |
| Human | Tumor | type II | NM_001297562.2 | MVSHRYPRIQSIKVQFTEYKKEKGFILTSQKEDEIMKVQ | 10 |
| OX40-L | necrosis | trans- | | NNSVIINCDGFYLISLKGYFSQEVNISLHYQKDEEPLFQ | |
| | factor | membrane | | LKKVRSVNSLMVASLTYKDKVYLNVTTDNTSLDDFHVNG | |
| | ligand | poly- | | GELILIHQNPGEFCVL | |
| | superfamily | peptide | | | |
| | member 4 | | | | |
| Human | Tumor | type II | P01375 | MSTESMIRDVELAEEALPKKTGGPQGSRRCLFLSLFSFL | 11 |
| TNF-α | necrosis | trans- | | IVAGATTLFCLLHFGVIGPQREEFPRDLSLISPLAQAVR | |
| | factor | membrane | | SSSRTPSDKPVAHVVANPQAEGQLQWLNRRANALLANGV | |
| | | poly- | | ELRDNQLVVPSEGLYLIYSQVLFKGQGCPSTHVLLTHTI | |
| | | peptide | | SRIAVSYQTKVNLLSAIKSPCQRETPEGAEAKPWYEPIY | |
| | | | | LGGVFQLEKGDRLSAEINRPDYLDFAESGQVYFGIIAL | |
| Human | A | type II | O75888 | MPASSPFLLAPKGPPGNMGGPVREPALSVALWLSWGAAL | 12 |
| APRIL | proliferation- | trans- | | GAVACAMALLTQQTELQSLRREVSRLQGTGGPSQNGEGY | |
| | inducing | membrane | | PWQSLPEQSSDALEAWENGERSRKRRAVLTQKQKKQHSV | |
| | ligand | poly- | | LHLVPINATSKDDSDVTEVMWQPALRRGRGLQAQGYGVR | |
| | | peptide | | IQDAGVYLLYSQVLFQDVTFTMGQVVSREGQGRQETLFR | |
| | | | | CIRSMPSHPDRAYNSCYSAGVFHLHQGDILSVIIPRARA | |
| | | | | KLNLSPHGTFLGFVKL | |
| Human | Transmembrane | type III | O14836 | MSGLGRSRRGGRSRVDQEERFPQGLWTGVAMRSCPEEQY | 13 |
| TACI | activator | trans- | | WDPLLGTCMSCKTICNHQSQRTCAAFCRSLSCRKEQGKF | |
| | and CAML | membrane | | YDHLLRDCISCASICGQHPKQCAYFCENKLRSPVNLPPE | |
| | interactor | poly- | | LRRQRSGEVENNSDNSGRYQGLEHRGSEASPALPGLKLS | |
| | | peptide | | ADQVALVYSTLGLCLCAVLCCFLVAVACFLKKRGDPCSC | |
| | | | | QPRSRPRQSPAKSSQDHAMEAGSPVSTSPEPVETCSFCF | |
| | | | | PECRAPTQESAVTPGTPDPTCAGRWGCHTRTTVLQPCPH | |
| | | | | IPDSGLGIVCVPAQEGGPGA | |
| Murine | Transmembrane | type III | Q9ET35 | MAMAFCPKDQYWDSSRKSCVSCALTCSQRSQRTCTDFCK | 14 |
| TACI | activator | trans- | | FINCRKEQGRYYDHLLGACVSCDSTCTQHPQQCAHFCEK | |
| | and CAML | membrane | | RPRSQANLQPELGRPQAGEVEVRSDNSGRHQGSEHGPGL | |
| | interactor | poly- | | RLSSDQLTLYCTLGVCLCAIFCCFLVALASFLRRRGEPL | |
| | | peptide | | PSQPAGPRGSQANSPHAHRPVTEACDEVTASPQPVETCS | |
| | | | | FCFPERSSPTQESAPRSLGIHGFAGTAAPQPCMRATVGG | |
| | | | | LGVLRASTGDARPAT | |
| Human | CD160 | GPI- | O95971 | MLLEPGRGCCALAILLAIVDIQSGGCINITSSASQEGTR | 15 |
| CD160 | antigen | anchored | | LNLICTVWHKKEEAEGFVVFLCKDRSGDCSPETSLKQLR | |
| | | poly- | | LKRDPGIDGVGEISSQLMFTISQVTPLHSGTYQCCARSQ | |
| | | peptide | | KSGIRLQGHFFSILFTETGNYTVTGLKQRQHLEFSHNEG | |
| | | | | TLSSGFLQEKVWVMLVTSLVALQAL | |
| Human | Signaling | GPI- | P09326 | MCSRGWDSCLALELLLLPLSLLVTSIQGHLVHMTVVSGS | 16 |
| CD48 | lymphocytic | anchored | | NVTLNISESLPENYKQLTWFYTFDQKIVEWDSRKSKYFE | |
| | activation | poly- | | SKFKGRVRLDPQSGALYISKVQKEDNSTYIMRVLKKTGN | |
| | molecule 2 | peptide | | EQEWKIKLQVLDPVPKPVIKIEKIEDMDDNCYLKLSCVI | |
| | | | | PGESVNYTWYGDKRPFPKELQNSVLETTLMPHNYSRCYT | |
| | | | | CQVSNSVSSKNGTVCLSPPCTLARSFGVEWIASWLVVTV | |
| | | | | PTILGLLLT | |
| Human | Interferon | Secreted | P01574 | MTNKCLLQIALLLCFSTTALSMSYNLLGFLQRSSNFQCQ | 17 |
| IFN-β | β | poly- | | KLLWQLNGRLEYCLKDRMNFDIPEEIKQLQQFQKEDAAL | |
| | | peptide; | | TIYEMLQNIFAIFRQDSSSTGWNETIVENLLANVYHQIN | |
| | | cytokine | | HLKTVLEEKLEKEDFTRGKLMSSLHLKRYYGRILHYLKA | |
| | | | | KEYSHCAWTIVRVEILRNFYFINRLTGYLRN | |
| Human | Lymphotoxin | Secreted | P01374 | MTPPERLFLPRVCGTTLHLLLLGLLLVLLPGAQGLPGVG | 18 |
| LT-α | alpha | poly- | | LTPSAAQTARQHPKMHLAHSTLKPAAHLIGDPSKQNSLL | |
| | | peptide | | WRANTDRAFLQDGFSLSNNSLLVPTSGIYFVYSQVVFSG | |
| | | | | KAYSPKATSSPLYLAHEVQLFSSQYPFHVPLLSSQKMVY | |
| | | | | PGLQEPWLHSMYHGAAFQLTQGDQLSTHTDGIPHLVLSP | |
| | | | | STVFFGAFAL | |
| Human | C-X-C | Multi- | P61073 | MEGISIYTSDNYTEEMGSGDYDSMKEPCFREENANFNKI | 19 |
| CXCR-4 | chemokine | pass | | FLPTIYSIIFLTGIVGNGLVILVMGYQKKLRSMTDKYRL | |
| | receptor | trans- | | HLSVADLLFVITLPFWAVDAVANWYFGNFLCKAVHVIYT | |
| | type 4 | membrane | | VNLYSSVLILAFISLDRYLAIVHATNSQRPRKLLAEKVV | |
| | | poly- | | YVGVWIPALLLTIPDFIFANVSEADDRYICDRFYPNDLW | |
| | | peptide; | | VVVFQFQHIMVGLILPGIVILSCYCIIISKLSHSKGHQK | |
| | | receptor | | RKALKTTVILILAFFACWLPYYIGISIDSFILLEIIKQG | |
| | | | | CEFENTVHKWISITEALAFFHCCLNPILYAFLGAKFKTS | |
| | | | | AQHALTSVSRGSSLKILSKGKRGGHSSVSTESESSSFHS | |
| | | | | S | |
| Human | Interferon | Cytokine | P01579 | MKYTSYILAFQLCIVLGSLGCYCQDPYVKEAENLKKYFN | 20 |
| IFNγ | Gamma | | | AGHSDVADNGTLFLGILKNWKEESDRKIMQSQIVSFYFK | |
| | | | | LFKNFKDDQSIQKSVETIKEDMNVKFFNSNKKKRDDFEK | |
| | | | | LTNYSVTDLNVQRKAIHELIQVMAELSPAAKTGKRKRSQ | |
| | | | | MLFRGRRASQ | |
| Human | Programmed | Immune | NM_014143.4 | MRIFAVFIFMTYWHLLNAFTVTVPKDLYVVEYGSNMTIE | 21 |
| PD-L1 | cell | check- | | CKFPVEKQLDLAALIVYWEMEDKNIIQFVHGEEDLKVQH | |
| | death 1 | point | | SSYRQRARLLKDQLSLGNAALQITDVKLQDAGVYRCMIS | |
| | ligand 1 | peptide | | YGGADYKRITVKVNAPYNKINQRILVVDPVTSEHELTCQ | |
| | | | | AEGYPKAEVIWTSSDHQVLSGKTTTTNSKREEKLFNVTS | |
| | | | | TLRINTTTNEIFYCTFRRLDPEENHTAELVIPELPLAHP | |
| | | | | PNERTHLVILGAILLCLGVALTFIFRLRKGRMMDVKKCG | |
| | | | | IQDTNSKKQSDTHLEET | |
| Human | Programmed | Immune | NP_005009.2 | MQIPQAPWPVVWAVLQLGWRPGWFLDSPDRPWNPPTFSP | 22 |
| PD-1 | cell | check- | | ALLVVTEGDNATFTCSFSNTSESFVLNWYRMSPSNQTDK | |
| | death | point | | LAAFPEDRSQPGQDCRFRVTQLPNGRDFHMSVVRARRND | |
| | protein 1 | peptide | | SGTYLCGAISLAPKAQIKESLRAELRVTERRAEVPTAHP | |
| | | | | SPSPRPAGQFQTLVVGVVGGLLGSLVLLVWVLAVICSRA | |
| | | | | ARGTIGARRTGQPLKEDPSAVPVFSVDYGELDFQWREKT | |
| | | | | PEPPVPCVPEQTEYATIVFPSGMGTSSPARRGSADGPRS | |
| | | | | AQPLRPEDGHCSWPL | |
| Human | C-X-C | chemokine | P48061 | MNAKVVVVLVLVLTALCLSDGKPVSLSYRCPCRFFESHV | 23 |
| CXCL-12 | motif | | | ARANVKHLKILNTPNCALQIVARLKNNNRQVCIDPKLKW | |
| | chemokine | | | IQEYLEKALNKRFKM | |
| | 12 | | | | |
| Murine | C-X-C | chemokine | P40224 | MDAKVVAVLALVLAALCISDGKPVSLSYRCPCRFFESHI | 24 |
| CXCL-12 | motif | | | ARANVKHLKILNTPNCALQIVARLKNNNRQVCIDPKLKW | |
| | chemokine | | | IQEYLEKALNKRLKM | |
| | 12 | | | | |
| Human | Intercellular | adhesion | NP_000192.2 | MAPSSPRPALPALLVLLGALFPGPGNAQTSVSPSKVILP | 25 |
| ICAM-1 | adhesion | factor | | RGGSVLVTCSTSCDQPKLLGIETPLPKKELLLPGNNRKV | |
| | molecule 1 | | | YELSNVQEDSQPMCYSNCPDGQSTAKTFLTVYWTPERVE | |
| | | | | LAPLPSWQPVGKNLTLRCQVEGGAPRANLTVVLLRGEKE | |
| | | | | LKREPAVGEPAEVTTTVLVRRDHHGANFSCRTELDLRPQ | |
| | | | | GLELFENTSAPYQLQTFVLPATPPQLVSPRVLEVDTQGT | |
| | | | | VVCSLDGLFPVSEAQVHLALGDQRLNPTVTYGNDSFSAK | |
| | | | | ASVSVTAEDEGTQRLTCAVILGNQSQETLQTVTIYSFPA | |
| | | | | PNVILTKPEVSEGTEVTVKCEAHPRAKVTLNGVPAQPLG | |
| | | | | PRAQLLLKATPEDNGRSFSCSATLEVAGQLIHKNQTREL | |
| | | | | RVLYGPRLDERDCPGNWTWPENSQQTPMCQAWGNPLPEL | |
| | | | | KCLKDGTFPLPIGESVTVTRDLEGTYLCRARSTQGEVTR | |
| | | | | KVTVNVLSPRYEIVIITVVAAAVIMGTAGLSTYLYNRQR | |
| | | | | KIKKYRLQQAQKGTPMKPNTQATPP | |
| Murine | Intercellular | adhesion | NM_010493.3 | MASTRAKPTLPLLLALVTVVIPGPGDAQVSIHPREAFLP | 26 |
| ICAM-1 | adhesion | factor | | QGGSVQVNCSSSCKEDLSLGLETQWLKDELESGPNWKLF | |
| | molecule 1 | | | ELSEIGEDSSPLCFENCGTVQSSASATITVYSFPESVEL | |
| | | | | RPLPAWQQVGKDLTLRCHVDGGAPRTQLSAVLLRGEEIL | |
| | | | | SRQPVGGHPKDPKEITFTVLASRGDHGANFSCRTELDLR | |
| | | | | PQGLALFSNVSEARSLRTFDLPATIPKLDTPDLLEVGTQ | |
| | | | | QKLFCSLEGLFPASEARIYLELGGQMPTQESTNSSDSVS | |
| | | | | ATALVEVTEEFDRTLPLRCVLELADQILETQRTLTVYNF | |
| | | | | SAPVLTLSQLEVSEGSQVTVKCEAHSGSKVVLLSGVEPR | |
| | | | | PPTPQVQFTLNASSEDHKRSFFCSAALEVAGKFLFKNQT | |
| | | | | LELHVLYGPRLDETDCLGNWTWQEGSQQTLKCQAWGNPS | |
| | | | | PKMTCRRKADGALLPIGVVKSVKQEMNGTYVCHAFSSHG | |
| | | | | NVTRNVYLTVLYHSQNNWTIIILVPVLLVIVGLVMAASY | |
| | | | | VYNRQRKIRIYKLQKAQEEAIKLKGQAPPP | |

In some embodiments, the display polypeptide comprises an amino acid sequence according to any amino acid sequence of Table 2, or a sequence that is substantially similar to a sequence of Table 2 (e.g. 90%, 91%, 92%, 93%, 94%, 95%, 96%, 97%, 98%, 99% sequence identity). In some instances, the display polypeptide comprises an amino acid sequence comprising at least a portion having at least or about 50, 60, 70, 80, 90, 100, 150, 200, 250, 300, 350, 400, 450, 500, 550, 600, 650, 700, 750, 800, 850, 900, 950, or 1000 amino acid sequences of any sequence according to Table 2. In some embodiments, the display polypeptide comprises an amino acid sequence that has at least 85% sequence identity to an amino acid sequence according to SEQ ID NOs: 7-26. In some embodiments, the display polypeptide comprises an amino acid sequence that has at least 95% sequence identity to an amino acid sequence according to SEQ ID NOs: 7-26.

In some embodiments, the transmembrane domain anchors the recombinant fusion protein to a bilayer of the enveloped particle. In some embodiments, the transmembrane domain comprises the transmembrane domain of a Vesicular Stomatitis virus glycoprotein (VSV-G). In some embodiments, the recombinant fusion protein comprises the transmembrane domain and cytosolic domain of a Vesicular Stomatitis virus glycoprotein (VSV-G). In some embodiments, the transmembrane domain comprises the transmembrane domain of a Dengue E protein. In some embodiments, the recombinant fusion protein comprises the transmembrane domain and cytosolic domain of a Dengue E protein. In some embodiments, the transmembrane domain comprises the transmembrane domain of influenza Neuraminidase (NA). In some embodiments, the recombinant fusion protein comprises the transmembrane domain and cytosolic domain of influenza Neuraminidase (NA).

TABLE 3
Exemplary Transmembrane Domain Sequences

| | | SEQ ID |
|---|---|---|
| Domain | Amino Acid Sequence | NO: |
| VSV-G | IASFFFIIGLIIGLFLVLRVGI | 27 |
| Transmembrane | | |
| (TM) V1 | | |
| VSV-G | PIELVEGWFSSWKSSIASFFFI | 28 |
| Transmembrane | IGLIIGLFLVLRVGI | |
| (TM) V2 | | |
| VSV-G | DDESLFFGDTGLSKNPIELVEG | 29 |
| Transmembrane | WFSSWKSSIASFFFIIGLIIGL | |
| (TM) V3 | FLVLRVGIH | |
| VSV-G | GMLDSDLHLSSKAQVFEHPHIQ | 30 |
| Transmembrane | DAASQLPDDESLFFGDTGLSKN | |
| (TM) V4 | PIELVEGWFSSWKSSIASFFFI | |
| | IGLIIGLFLVLRVGI | |
| VSV-G | HLCIKLKHTKKRQIYTDIEMNR | 31 |
| Cytosolic Tail | LGK | |
| (CT) | | |
| Influenza | IITIGSVCMTIGMANLILQIGN | 32 |
| Neuraminidase | I | |
| TM (N1) | | |
| Influenza | LAIYSTVASSLVLVVSLGAISF | 33 |
| Hemagglutinin | W | |
| TM (H1) | | |
| Dengue E | AYGVLFSGVSWTMKIGIGILLT | 34 |
| Protein TM | WLGLNSRSTSLSMTCIAVGMVT | |
| | LYLGVMVQ | |
| HIV gp TM | FIMIVGGLVGLRIVFAVLSIV | 35 |

In some embodiments, the transmembrane domain comprises an amino acid sequence that has at least 85% sequence identity to an amino acid sequence according to SEQ ID NOs: 27-35. In some embodiments, the transmembrane domain comprises an amino acid sequence that has at least 95% sequence identity to an amino acid sequence according to SEQ ID NOs: 27-35.

In some embodiments, the transmembrane domain comprises an amino acid sequence of at least 75% sequence identity to an amino acid sequence according to SEQ ID NO: 27. In some embodiments, the transmembrane domain comprises an amino acid sequence of at least 80% sequence identity to an amino acid sequence according to SEQ ID NO: 27. In some embodiments, the transmembrane domain comprises an amino acid sequence of at least 85% sequence identity to an amino acid sequence according to SEQ ID NO: 27. In some embodiments, the transmembrane domain comprises an amino acid sequence of at least 90% sequence identity to an amino acid sequence according to SEQ ID NO: 27. In some embodiments, the transmembrane domain comprises an amino acid sequence of at least 95% sequence identity to an amino acid sequence according to SEQ ID NO: 27. In some embodiments, the transmembrane domain comprises an amino acid sequence of at least 75% sequence homology to an amino acid sequence according to SEQ ID NO: 27. In some embodiments, the transmembrane domain comprises an amino acid sequence of at least 80% sequence homology to an amino acid sequence according to SEQ ID NO: 27. In some embodiments, the transmembrane domain comprises an amino acid sequence of at least 85% sequence homology to an amino acid sequence according to SEQ ID NO: 27. In some embodiments, the transmembrane domain comprises an amino acid sequence of at least 90% sequence homology to an amino acid sequence according to SEQ ID NO: 27. In some embodiments, the transmembrane domain comprises an amino acid sequence of at least 95% sequence homology to an amino acid sequence according to SEQ ID NO: 27. In some embodiments, the transmembrane domain comprises an amino acid sequence according to SEQ ID NO: 27.

In some embodiments, the transmembrane domain comprises an amino acid sequence of at least 75% sequence identity to an amino acid sequence according to SEQ ID NO: 28. In some embodiments, the transmembrane domain comprises an amino acid sequence of at least 80% sequence identity to an amino acid sequence according to SEQ ID NO: 28. In some embodiments, the transmembrane domain comprises an amino acid sequence of at least 85% sequence identity to an amino acid sequence according to SEQ ID NO: 28. In some embodiments, the transmembrane domain comprises an amino acid sequence of at least 90% sequence identity to an amino acid sequence according to SEQ ID NO: 28. In some embodiments, the transmembrane domain comprises an amino acid sequence of at least 95% sequence identity to an amino acid sequence according to SEQ ID NO: 28. In some embodiments, the transmembrane domain comprises an amino acid sequence of at least 75% sequence homology to an amino acid sequence according to SEQ ID NO: 28. In some embodiments, the transmembrane domain comprises an amino acid sequence of at least 80% sequence homology to an amino acid sequence according to SEQ ID NO: 28. In some embodiments, the transmembrane domain comprises an amino acid sequence of at least 85% sequence homology to an amino acid sequence according to SEQ ID NO: 28. In some embodiments, the transmembrane domain comprises an amino acid sequence of at least 90% sequence homology to an amino acid sequence according to SEQ ID NO: 28. In some embodiments, the transmembrane domain comprises an amino acid sequence of at least 95% sequence homology to an amino acid sequence according to SEQ ID NO: 28. In some embodiments, the transmembrane domain comprises an amino acid sequence according to SEQ ID NO: 28.

In some embodiments, the transmembrane domain comprises an amino acid sequence of at least 75% sequence identity to an amino acid sequence according to SEQ ID NO: 29. In some embodiments, the transmembrane domain comprises an amino acid sequence of at least 80% sequence identity to an amino acid sequence according to SEQ ID NO: 29. In some embodiments, the transmembrane domain comprises an amino acid sequence of at least 85% sequence identity to an amino acid sequence according to SEQ ID NO: 29. In some embodiments, the transmembrane domain comprises an amino acid sequence of at least 90% sequence identity to an amino acid sequence according to SEQ ID NO: 29. In some embodiments, the transmembrane domain comprises an amino acid sequence of at least 95% sequence identity to an amino acid sequence according to SEQ ID NO: 29. In some embodiments, the transmembrane domain comprises an amino acid sequence of at least 75% sequence homology to an amino acid sequence according to SEQ ID NO: 29. In some embodiments, the transmembrane domain comprises an amino acid sequence of at least 80% sequence homology to an amino acid sequence according to SEQ ID NO: 29. In some embodiments, the transmembrane domain comprises an amino acid sequence of at least 85% sequence homology to an amino acid sequence according to SEQ ID NO: 29. In some embodiments, the transmembrane domain comprises an amino acid sequence of at least 90% sequence homology to an amino acid sequence according to SEQ ID NO: 29. In some embodiments, the transmembrane domain comprises an amino acid sequence of at least 95% sequence homology to an amino acid sequence according to SEQ ID NO: 29. In some embodiments, the transmembrane domain comprises an amino acid sequence according to SEQ ID NO: 29.

In some embodiments, the transmembrane domain comprises an amino acid sequence of at least 75% sequence identity to an amino acid sequence according to SEQ ID NO: 30. In some embodiments, the transmembrane domain comprises an amino acid sequence of at least 80% sequence identity to an amino acid sequence according to SEQ ID NO: 30. In some embodiments, the transmembrane domain comprises an amino acid sequence of at least 85% sequence identity to an amino acid sequence according to SEQ ID NO: 30. In some embodiments, the transmembrane domain comprises an amino acid sequence of at least 90% sequence identity to an amino acid sequence according to SEQ ID NO: 30. In some embodiments, the transmembrane domain comprises an amino acid sequence of at least 95% sequence identity to an amino acid sequence according to SEQ ID NO: 30. In some embodiments, the transmembrane domain comprises an amino acid sequence of at least 75% sequence homology to an amino acid sequence according to SEQ ID NO: 30. In some embodiments, the transmembrane domain comprises an amino acid sequence of at least 80% sequence homology to an amino acid sequence according to SEQ ID NO: 30. In some embodiments, the transmembrane domain comprises an amino acid sequence of at least 85% sequence homology to an amino acid sequence according to SEQ ID NO: 30. In some embodiments, the transmembrane domain comprises an amino acid sequence of at least 90% sequence homology to an amino acid sequence according to SEQ ID NO: 30. In some embodiments, the transmembrane domain comprises an amino acid sequence of at least 95% sequence homology to an amino acid sequence according to SEQ ID NO: 30. In some embodiments, the transmembrane domain comprises an amino acid sequence according to SEQ ID NO: 30.

In some embodiments, the transmembrane domain comprises an amino acid sequence of at least 75% sequence identity to an amino acid sequence according to SEQ ID NO: 31. In some embodiments, the transmembrane domain comprises an amino acid sequence of at least 80% sequence identity to an amino acid sequence according to SEQ ID NO: 31. In some embodiments, the transmembrane domain comprises an amino acid sequence of at least 85% sequence identity to an amino acid sequence according to SEQ ID NO: 31. In some embodiments, the transmembrane domain comprises an amino acid sequence of at least 90% sequence identity to an amino acid sequence according to SEQ ID NO: 31. In some embodiments, the transmembrane domain comprises an amino acid sequence of at least 95% sequence identity to an amino acid sequence according to SEQ ID NO: 31. In some embodiments, the transmembrane domain comprises an amino acid sequence of at least 75% sequence homology to an amino acid sequence according to SEQ ID NO: 31. In some embodiments, the transmembrane domain comprises an amino acid sequence of at least 80% sequence homology to an amino acid sequence according to SEQ ID NO: 31. In some embodiments, the transmembrane domain comprises an amino acid sequence of at least 85% sequence homology to an amino acid sequence according to SEQ ID NO: 31. In some embodiments, the transmembrane domain comprises an amino acid sequence of at least 90% sequence homology to an amino acid sequence according to SEQ ID NO: 31. In some embodiments, the transmembrane domain comprises an amino acid sequence of at least 95% sequence homology to an amino acid sequence according to SEQ ID NO: 31. In some embodiments, the transmembrane domain comprises an amino acid sequence according to SEQ ID NO: 31.

In some embodiments, the transmembrane domain comprises an amino acid sequence of at least 75% sequence identity to an amino acid sequence according to SEQ ID NO: 32. In some embodiments, the transmembrane domain comprises an amino acid sequence of at least 80% sequence identity to an amino acid sequence according to SEQ ID NO: 32. In some embodiments, the transmembrane domain comprises an amino acid sequence of at least 85% sequence identity to an amino acid sequence according to SEQ ID NO: 32. In some embodiments, the transmembrane domain comprises an amino acid sequence of at least 90% sequence identity to an amino acid sequence according to SEQ ID NO: 32. In some embodiments, the transmembrane domain comprises an amino acid sequence of at least 95% sequence identity to an amino acid sequence according to SEQ ID NO: 32. In some embodiments, the transmembrane domain comprises an amino acid sequence of at least 75% sequence homology to an amino acid sequence according to SEQ ID NO: 32. In some embodiments, the transmembrane domain comprises an amino acid sequence of at least 80% sequence homology to an amino acid sequence according to SEQ ID NO: 32. In some embodiments, the transmembrane domain comprises an amino acid sequence of at least 85% sequence homology to an amino acid sequence according to SEQ ID NO: 32. In some embodiments, the transmembrane domain comprises an amino acid sequence of at least 90% sequence homology to an amino acid sequence according to SEQ ID NO: 32. In some embodiments, the transmembrane domain comprises an amino acid sequence of at least 95% sequence homology to an amino acid sequence according to SEQ ID NO: 32. In some embodiments, the transmembrane domain comprises an amino acid sequence according to SEQ ID NO: 32.

In some embodiments, the transmembrane domain comprises an amino acid sequence of at least 75% sequence identity to an amino acid sequence according to SEQ ID NO: 33. In some embodiments, the transmembrane domain comprises an amino acid sequence of at least 80% sequence identity to an amino acid sequence according to SEQ ID NO: 33. In some embodiments, the transmembrane domain comprises an amino acid sequence of at least 85% sequence identity to an amino acid sequence according to SEQ ID NO: 33. In some embodiments, the transmembrane domain comprises an amino acid sequence of at least 90% sequence identity to an amino acid sequence according to SEQ ID NO: 33. In some embodiments, the transmembrane domain comprises an amino acid sequence of at least 95% sequence identity to an amino acid sequence according to SEQ ID NO: 33. In some embodiments, the transmembrane domain comprises an amino acid sequence of at least 75% sequence homology to an amino acid sequence according to SEQ ID NO: 33. In some embodiments, the transmembrane domain comprises an amino acid sequence of at least 80% sequence homology to an amino acid sequence according to SEQ ID NO: 33. In some embodiments, the transmembrane domain comprises an amino acid sequence of at least 85% sequence homology to an amino acid sequence according to SEQ ID NO: 33. In some embodiments, the transmembrane domain comprises an amino acid sequence of at least 90% sequence homology to an amino acid sequence according to SEQ ID NO: 33. In some embodiments, the transmembrane domain comprises an amino acid sequence of at least 95% sequence homology to an amino acid sequence according to SEQ ID NO: 33. In some embodiments, the transmembrane domain comprises an amino acid sequence according to SEQ ID NO: 33.

In some embodiments, the transmembrane domain comprises an amino acid sequence of at least 75% sequence identity to an amino acid sequence according to SEQ ID NO: 34. In some embodiments, the transmembrane domain comprises an amino acid sequence of at least 80% sequence identity to an amino acid sequence according to SEQ ID NO: 34. In some embodiments, the transmembrane domain comprises an amino acid sequence of at least 85% sequence identity to an amino acid sequence according to SEQ ID NO: 34. In some embodiments, the transmembrane domain comprises an amino acid sequence of at least 90% sequence identity to an amino acid sequence according to SEQ ID NO: 34. In some embodiments, the transmembrane domain comprises an amino acid sequence of at least 95% sequence identity to an amino acid sequence according to SEQ ID NO: 34. In some embodiments, the transmembrane domain comprises an amino acid sequence of at least 75% sequence homology to an amino acid sequence according to SEQ ID NO: 34. In some embodiments, the transmembrane domain comprises an amino acid sequence of at least 80% sequence homology to an amino acid sequence according to SEQ ID NO: 34. In some embodiments, the transmembrane domain comprises an amino acid sequence of at least 85% sequence homology to an amino acid sequence according to SEQ ID NO: 34. In some embodiments, the transmembrane domain comprises an amino acid sequence of at least 90% sequence homology to an amino acid sequence according to SEQ ID NO: 34. In some embodiments, the transmembrane domain comprises an amino acid sequence of at least 95% sequence homology to an amino acid sequence according to SEQ ID NO: 34. In some embodiments, the transmembrane domain comprises an amino acid sequence according to SEQ ID NO: 34.

In some embodiments, the transmembrane domain comprises an amino acid sequence of at least 75% sequence identity to an amino acid sequence according to SEQ ID NO: 35. In some embodiments, the transmembrane domain comprises an amino acid sequence of at least 80% sequence identity to an amino acid sequence according to SEQ ID NO: 35. In some embodiments, the transmembrane domain comprises an amino acid sequence of at least 85% sequence identity to an amino acid sequence according to SEQ ID NO: 35. In some embodiments, the transmembrane domain comprises an amino acid sequence of at least 90% sequence identity to an amino acid sequence according to SEQ ID NO: 35. In some embodiments, the transmembrane domain comprises an amino acid sequence of at least 95% sequence identity to an amino acid sequence according to SEQ ID NO: 35. In some embodiments, the transmembrane domain comprises an amino acid sequence of at least 75% sequence homology to an amino acid sequence according to SEQ ID NO: 35. In some embodiments, the transmembrane domain comprises an amino acid sequence of at least 80% sequence homology to an amino acid sequence according to SEQ ID NO: 35. In some embodiments, the transmembrane domain comprises an amino acid sequence of at least 85% sequence homology to an amino acid sequence according to SEQ ID NO: 35. In some embodiments, the transmembrane domain comprises an amino acid sequence of at least 90% sequence homology to an amino acid sequence according to SEQ ID NO: 35. In some embodiments, the transmembrane domain comprises an amino acid sequence of at least 95% sequence homology to an amino acid sequence according to SEQ ID NO: 35. In some embodiments, the transmembrane domain comprises an amino acid sequence according to SEQ ID NO: 35.

In some embodiments, the transmembrane domain comprises an amino acid sequence disclosed in Table 3, or an amino acid sequence that is substantially identical to an amino acid sequence in Table 3 (e.g. 80%, 85%, 90%, 95%, 96%, 97%, 98%, 99% sequence identity). In some instances, the transmembrane domain comprises an amino acid sequence comprising at least a portion having at least or about 10, 20, 30, 40, 50, 60, 70, 80, 90, 100, 110, 120, 130 amino acid sequences of any sequence according to Table 3.

### Valencies

In some embodiments, when the recombinant fusion protein is expressed on the surface of the enveloped particle the recombinant fusion protein is expressed at a valency of about 10 copies on the surface of the enveloped particle. In some embodiments, when the recombinant fusion protein is expressed on the surface of the enveloped particle the recombinant fusion protein is expressed at a valency of about 10 to 15 copies on the surface of the enveloped particle. In some embodiments, when the recombinant fusion protein is expressed on the surface of the enveloped particle the recombinant fusion protein is expressed at a valency of at least about 25 copies on the surface of the enveloped particle. In some embodiments, when the recombinant fusion protein is expressed on the surface of the enveloped particle the recombinant fusion protein is expressed at a valency of at least about 50 copies on the surface of the enveloped particle. In some embodiments, when the recombinant fusion protein is expressed on the surface of the enveloped particle the recombinant fusion protein is expressed at a valency of at least about 100 copies on the surface of the enveloped particle. In some embodiments, when the recombinant fusion protein is expressed on the surface of the enveloped particle the recombinant fusion protein is expressed at a valency of at least about 200 copies on the surface of the enveloped particle. In some embodiments, when the recombinant fusion protein is expressed on the surface of the enveloped particle the recombinant fusion protein is expressed at a valency of at least about 400 copies on the surface of the enveloped particle. In some embodiments, when the recombinant fusion protein is expressed on the surface of the enveloped particle the recombinant fusion protein is expressed at a valency of at least about 600 copies on the surface of the enveloped particle. In some embodiments, when the recombinant fusion protein is expressed on the surface of the enveloped particle the recombinant fusion protein is expressed at least about 1000 copies on the surface of the enveloped particle. In some embodiments, when the recombinant fusion protein is expressed on the surface of the enveloped particle the recombinant fusion protein is expressed at least about 1500 copies on the surface of the enveloped particle. In some embodiments, when the recombinant fusion protein is expressed on the surface of the enveloped particle the recombinant fusion protein is expressed at least about 2000 copies on the surface of the enveloped particle.

Disclosed herein, in certain embodiments, are an enveloped particle comprising: a) a first recombinant fusion protein that is displayed in an oligomeric format on the surface of the enveloped particle wherein the first recombinant fusion protein comprises a transmembrane domain, a display polypeptide, and a human oligomerization domain; and b) a second recombinant fusion protein that is displayed in a monomeric format on the surface of the enveloped particle wherein the second recombinant fusion protein comprises a transmembrane domain and a display polypeptide.

In some embodiments, when the first recombinant fusion protein is expressed on the surface of the enveloped particle the first recombinant fusion protein is expressed at a valency of about 10 copies on the surface of the enveloped particle. In some embodiments, when the first recombinant fusion protein is expressed on the surface of the enveloped particle the first recombinant fusion protein is expressed at a valency of about 10 to 15 copies on the surface of the enveloped particle. In some embodiments, when the first recombinant fusion protein is expressed on the surface of the enveloped particle the first recombinant fusion protein is expressed at a valency of at least about 25 copies on the surface of the enveloped particle. In some embodiments, when the first recombinant fusion protein is expressed on the surface of the enveloped particle the first recombinant fusion protein is expressed at a valency of at least about 50 copies on the surface of the enveloped particle. In some embodiments, when the first recombinant fusion protein is expressed on the surface of the enveloped particle the first recombinant fusion protein is expressed at a valency of at least about 100 copies on the surface of the enveloped particle. In some embodiments, when the first recombinant fusion protein is expressed on the surface of the enveloped particle the first recombinant fusion protein is expressed at a valency of at least about 200 copies on the surface of the enveloped particle. In some embodiments, when the first recombinant fusion protein is expressed on the surface of the enveloped particle the first recombinant fusion protein is expressed at a valency of at least about 400 copies on the surface of the enveloped particle. In some embodiments, when the first recombinant fusion protein is expressed on the surface of the enveloped particle the first recombinant fusion protein is expressed at a valency of at least about 600 copies on the surface of the enveloped particle. In some embodiments, when the first recombinant fusion protein is expressed on the surface of the enveloped particle the first recombinant fusion protein is expressed on the surface of the enveloped particle at a valency of at least about 1000 copies on the surface of the enveloped particle.

In some embodiments, when the second recombinant fusion protein is expressed on the surface of the enveloped particle the second recombinant fusion protein is expressed at a valency of about 10 copies on the surface of the enveloped particle. In some embodiments, when the second recombinant fusion protein is expressed on the surface of the enveloped particle the second recombinant fusion protein is expressed at a valency of about 10 to 15 copies on the surface of the enveloped particle. In some embodiments, wherein when the second recombinant fusion protein is expressed on the surface of the enveloped particle the second recombinant fusion protein is expressed at a valency of at least about 25 copies on the surface of the enveloped particle. In some embodiments, when the second recombinant fusion protein is expressed on the surface of the enveloped particle the second recombinant fusion protein is expressed at a valency of at least about 50 copies on the surface of the enveloped particle. In some embodiments, when the second recombinant fusion protein is expressed on the surface of the enveloped particle the second recombinant fusion protein is expressed at a valency of at least about 100 copies on the surface of the enveloped particle. In some embodiments, when the second recombinant fusion protein is expressed on the surface of the enveloped particle the second recombinant fusion protein is expressed at a valency of at least about 200 copies on the surface of the enveloped particle. In some embodiments, when the second recombinant fusion protein is expressed on the surface of the enveloped particle the second recombinant fusion protein is expressed at a valency of at least about 400 copies on the surface of the enveloped particle. In some embodiments, when the second recombinant fusion protein is expressed on the surface of the enveloped particle the second recombinant fusion protein is expressed at a valency of at least about 600 copies on the surface of the enveloped particle. In some embodiments, when the second recombinant fusion protein is expressed on the surface of the enveloped particle the second recombinant fusion protein is expressed on the surface of the enveloped particle at a valency of at least about 1000 copies on the surface of the enveloped particle. when the second recombinant fusion protein is expressed on the surface of the enveloped particle the second recombinant fusion protein is expressed on the surface of the enveloped particle at a valency of at least about 2000 copies on the surface of the enveloped particle.

### Enveloped Particles

Disclosed herein are enveloped particles comprising the recombinant fusion proteins described herein. In some embodiments, the enveloped particle comprises a viral-like particle. In some embodiments, the enveloped particle comprises an enveloped virus. In some embodiments, the enveloped particle comprises an extracellular vesicle. In some embodiments, the extracellular vesicle comprises an exosome or an ectosome.

Disclosed herein are enveloped particles comprising: (a) a first recombinant fusion protein that is displayed in an oligomeric format on the surface of the enveloped particle wherein the first recombinant fusion protein comprises a transmembrane domain, a display polypeptide, and an oligomerization domain; and (b) a second recombinant fusion protein that is displayed in a monomeric format on the surface of the enveloped particle wherein the second recombinant fusion protein comprises a transmembrane domain and a display polypeptide.

Disclosed herein are enveloped particles comprising: (a) a first recombinant fusion protein that is displayed in a first oligomeric format on a surface of an enveloped particle, wherein the first recombinant fusion protein comprises a transmembrane domain, a display polypeptide, and a human oligomerization domain; and (b) a second recombinant fusion protein that is displayed in a second oligomeric format, wherein the second recombinant fusion protein comprises a transmembrane domain and a display polypeptide.

In some embodiments, the human oligomerization domain comprises a human coiled coil motif. In some embodiments, the human oligomerization domain is a dimerization domain. In some embodiments, the human oligomerization domain comprises a human fibrinogen-like protein 1 coiled coil (hFcc) domain. In some embodiments, the human oligomerization domain is a trimerization domain. In some embodiments, the human oligomerization domain comprises a LINE-1 retrotransposon orf1p coiled coil (L1cc) domain. In some embodiments, the human oligomerization domain comprises a human lung surfactant protein D coiled coil (hLSPDcc) domain. In some embodiments, the human oligomerization domain comprises a human tetranectin coiled coil (hTETNcc) domain. In some embodiments, the human oligomerization domain is a tetramerization domain. In some embodiments, the human oligomerization domain comprises a human guanylate-binding protein 1 coiled coil (hGBP1cc) domain.

In some embodiments, the oligomerization domain comprises an amino acid sequence disclosed in Table 1, or an amino acid sequence that is substantially identical to an amino acid sequence in Table 1 (e.g. 80%, 85%, 90%, 95%, 96%, 97%, 98%, 99% sequence identity). In some instances, the oligomerization domain comprises an amino acid sequence comprising at least a portion having at least or about 10, 20, 30, 40, 50, 60, 70, 80, 90, 100, 110, 120, 130 amino acid sequences of any sequence according to Table 1. In some embodiments, the human oligomerization domain comprises an amino acid sequence that has at least 85% sequence identity to an amino acid sequence according to SEQ ID NOs: 1-6. In some embodiments, the human oligomerization domain comprises an amino acid sequence that has at least 95% sequence identity to an amino acid sequence according to SEQ ID NOs: 1-6.

In some embodiments, when the first recombinant fusion protein is expressed on the surface of the enveloped particle, the human oligomerization domain is outside of the enveloped particle. In some embodiments, when the first recombinant fusion protein is expressed on the surface of the enveloped particle, the human oligomerization domain is outside of the enveloped particle and adjacent to a signal peptide. In some embodiments, when the first recombinant fusion protein is expressed on the surface of the enveloped particle, the human oligomerization domain is inside of the enveloped particle. In some embodiments, when the first recombinant fusion protein is expressed on the surface of the enveloped particle, the human oligomerization domain is inside of the enveloped particle and adjacent to the transmembrane domain.

In some embodiments, the first recombinant fusion protein further comprises a signal peptide. In some embodiments, domains of the first recombinant fusion protein are arranged from the N-terminus to the C-terminus in the following orders: a) signal peptide, display polypeptide, human oligomerization domain, and transmembrane domain; b) signal peptide, display polypeptide, transmembrane domain, and human oligomerization domain; or c) signal peptide, human oligomerization domain, display peptide, and transmembrane domain. In some embodiments, the first recombinant fusion protein further comprises a cytosolic domain. In some embodiments, domains of the first recombinant fusion protein are arranged from the N-terminus to the C-terminus in the following orders: a) signal peptide, display polypeptide, human oligomerization domain, transmembrane domain, and cytosolic domain; b) signal peptide, display polypeptide, transmembrane domain, human oligomerization domain, and cytosolic domain; or c) signal peptide, human oligomerization domain, display peptide, transmembrane domain, and cytosolic domain.

In some embodiments, the enveloped particle comprises an enveloped viral-like particle, an enveloped virus, an extracellular vesicle. In some embodiments, the extracellular vesicle comprises an exosome. In some embodiments, the extracellular vesicle comprises an ectosome. In some embodiments, the first recombinant fusion protein and the second recombinant fusion protein have the same display polypeptide. In some embodiments, the first recombinant fusion protein and the second recombinant fusion protein have different display polypeptides. In some embodiments, the display polypeptide targets a cell surface protein that is oligomerized on a target cell for agonistic function. In some embodiments, the display polypeptide targets a cell surface protein that is oligomerized on a target cell for antagonistic function. In some embodiments, the cell surface protein is dimerized and the human oligomerization domain is a dimerization domain. In some embodiments, the cell surface protein is trimerized and the human oligomerization domain is a trimerization domain. In some embodiments, the cell surface protein is tetramerized and the human oligomerization domain is a tetramerization domain.

In some embodiments, the display polypeptide comprises a type I transmembrane polypeptide, a type II transmembrane polypeptide, a type III transmembrane polypeptide, a GPI-anchored polypeptide, a secreted polypeptide, or a multi-pass transmembrane polypeptide. In some embodiments, the type I transmembrane polypeptide comprises ACE2, TIGIT, or LAG-3. In some embodiments, the type II transmembrane polypeptide comprises OX40-L, TNF-α, or APRIL. In some embodiments, the type III transmembrane polypeptide comprises TACI. In some embodiments, the GPI-anchored polypeptide comprises CD160 or CD48. In some embodiments, the secreted polypeptide comprises IFN-β or LT-α. In some embodiments, the multi-pass transmembrane polypeptide comprises CXCR-4. In some embodiments, the display polypeptide comprises an antibody, a receptor polypeptide, a cytokine, an immune checkpoint polypeptide, a chemokine, or an adhesion factor. In some embodiments, the receptor polypeptide comprises ACE2 or CXCR-4. In some embodiments, the cytokine comprises IFN-β or IFNγ. In some embodiments, the immune checkpoint polypeptide comprises PD-L1 or PD-1. In some embodiments, the chemokine comprises CXCL-12. In some embodiments, the adhesion factor comprises ICAM-1.

In some embodiments, the display polypeptide comprises an amino acid sequence according to any amino acid sequence of Table 2, or a sequence that is substantially similar to a sequence of Table 2 (e.g. 90%, 91%, 92%, 93%, 94%, 95%, 96%, 97%, 98%, 99% sequence identity). In some instances, the display polypeptide comprises an amino acid sequence comprising at least a portion having at least or about 50, 60, 70, 80, 90, 100, 150, 200, 250, 300, 350, 400, 450, 500, 550, 600, 650, 700, 750, 800, 850, 900, 950, or 1000 amino acid sequences of any sequence according to Table 2. In some embodiments, the display polypeptide comprises an amino acid sequence that has at least 85% sequence identity to an amino acid sequence according to SEQ ID NOs: 7-26. In some embodiments, the display polypeptide comprises an amino acid sequence that has at least 95% sequence identity to an amino acid sequence according to SEQ ID NOs: 7-26.

In some embodiments, the first recombinant fusion protein and the second recombinant fusion protein have the same transmembrane domains. In some embodiments, the first recombinant fusion protein and the second recombinant fusion protein have different transmembrane domains. In some embodiments, the transmembrane domain anchors the recombinant fusion protein to a bilayer of the enveloped particle. In some embodiments, the transmembrane domain comprises the transmembrane domain of a Vesicular Stomatitis virus glycoprotein (VSV-G). In some embodiments, the first recombinant fusion protein comprises the transmembrane domain and cytosolic domain of a Vesicular Stomatitis virus glycoprotein (VSV-G). In some embodiments, the transmembrane domain comprises the transmembrane domain of a Dengue E protein. In some embodiments, the first recombinant fusion protein comprises the transmembrane domain and cytosolic domain of a Dengue E protein. In some embodiments, the transmembrane domain comprises the transmembrane domain of influenza Neuraminidase (NA). In some embodiments, the first recombinant fusion protein comprises the transmembrane domain and cytosolic domain of influenza Neuraminidase (NA). In some embodiments, the transmembrane domain comprises an amino acid sequence that has at least 85% sequence identity to an amino acid sequence according to SEQ ID NOs: 27-35. In some embodiments, the transmembrane domain comprises an amino acid sequence that has at least 95% sequence identity to an amino acid sequence according to SEQ ID NOs: 27-35.

### Methods of Use

Disclosed herein, in certain embodiments, are a method of agonizing or antagonizing a cell that has an oligomerized cell surface protein comprising contacting the cell with an enveloped particle comprising a first recombinant fusion protein that is displayed in an oligomeric format on the surface of the enveloped particle, wherein the first recombinant fusion protein comprises a transmembrane domain, a display polypeptide, and a human oligomerization domain. In some embodiments, the enveloped particle further comprises a second recombinant fusion protein that is displayed in a monomeric format on the surface of the enveloped particle, wherein the second recombinant fusion protein comprises a transmembrane domain and a display polypeptide. In some embodiments, the human oligomerization domain comprises a human coiled coil motif. In some embodiments, the human oligomerization domain is a dimerization domain. In some embodiments, the human oligomerization domain comprises a human fibrinogen-like protein 1 coiled coil (hFcc) domain. In some embodiments, the human oligomerization domain is a trimerization domain. In some embodiments, the human oligomerization domain comprises a LINE-1 retrotransposon orf1p coiled coil (L1cc) domain. In some embodiments, the human oligomerization domain comprises a human lung surfactant protein D coiled coil (hLSPDcc) domain. In some embodiments, the human oligomerization domain comprises a human tetranectin coiled coil (hTETNcc) domain. In some embodiments, the human oligomerization domain is a tetramerization domain. In some embodiments, the human oligomerization domain comprises a human guanylate-binding protein 1 coiled coil (hGBP1cc) domain. In some embodiments, the human oligomerization domain comprises an amino acid sequence that has at least 85% sequence identity to an amino acid sequence according to SEQ ID NOs: 1-6. In some embodiments, the human oligomerization domain comprises an amino acid sequence that has at least 95% sequence identity to an amino acid sequence according to SEQ ID NOs: 1-6.

Disclosed herein, in certain embodiments, is a multivalent particle (MVP) comprising an enveloped particle, wherein the MVP displays at least about 10 copies of a peptide on a surface of the MVP, wherein at least one surface protein on the surface of the MVP comprises an oligomerized domain, wherein the peptide forms multivalent interactions with a cognate ligand on a target cell or a target virus.

Disclosed herein, in certain embodiments, is a method of using a multivalent particle (MVP), wherein the MVP displays an oligomerized peptide as an immunogen to generate protective immunity against a target antigen, wherein the target antigen is a bacterial antigen, a viral antigen, a fungal antigen, or a tumor antigens, wherein at least about 10 copies of the oligomerized peptide on a surface of the MVP, wherein at least one surface protein on the MVP comprises an oligomerized domain.

Disclosed herein, in certain embodiments, is a method of using a multivalent particle (MVP), wherein the MVP displays an oligomerized peptide as an immunogen to generate protective immunity against a target antigen, wherein the target antigen is a bacterial antigen, a viral antigen, a fungal antigen, or a tumor antigen, wherein the MVP comprises a double stranded ribonucleic viral genome as a toll-like receptor agonist adjuvant.

In some embodiments, when the first recombinant fusion protein is expressed on the surface of the enveloped particle, the human oligomerization domain is outside of the enveloped particle. In some embodiments, when the first recombinant fusion protein is expressed on the surface of the enveloped particle, the human oligomerization domain is outside of the enveloped particle and adjacent to a signal peptide. In some embodiments, when the first recombinant fusion protein is expressed on the surface of the enveloped particle, the human oligomerization domain is inside of the enveloped particle. In some embodiments, when the first recombinant fusion protein is expressed on the surface of the enveloped particle, the human oligomerization domain is inside of the enveloped particle and adjacent to the transmembrane domain.

In some embodiments, the first recombinant fusion protein comprises a signal peptide. In some embodiments, domains of the first recombinant fusion protein are arranged from the N-terminus to the C-terminus in the following orders: a) signal peptide, display polypeptide, human oligomerization domain, and transmembrane domain; b) signal peptide, display polypeptide, transmembrane domain, and human oligomerization domain; or c) signal peptide, human oligomerization domain, display peptide, and transmembrane domain. In some embodiments, the first recombinant fusion protein further comprises a cytosolic domain. In some embodiments, domains of the first recombinant fusion protein are arranged from the N-terminus to the C-terminus in the following orders: a) signal peptide, display polypeptide, human oligomerization domain, transmembrane domain, and cytosolic domain; b) signal peptide, display polypeptide, transmembrane domain, human oligomerization domain, and cytosolic domain; or c) signal peptide, human oligomerization domain, display peptide, transmembrane domain, and cytosolic domain.

In some embodiments, the enveloped particle comprises an enveloped viral-like particle, an enveloped virus, an extracellular vesicle. In some embodiments, the extracellular vesicle comprises an exosome. In some embodiments, the extracellular vesicle comprises an ectosome. In some embodiments, the first recombinant fusion protein and the second recombinant fusion protein have the same display polypeptide. In some embodiments, the first recombinant fusion protein and the second recombinant fusion protein have different display polypeptides. In some embodiments, the display polypeptide targets the cell surface protein that is oligomerized on a target cell for agonistic function. In some embodiments, the display polypeptide targets the cell surface protein that is oligomerized on a target cell for antagonistic function. In some embodiments, the cell surface protein is dimerized and the coiled coil oligomerization domain is a dimerization domain. In some embodiments, the cell surface protein is trimerized and the coiled coil oligomerization domain is a trimerization domain. In some embodiments, the cell surface protein is tetramerized and the coiled coil oligomerization domain is a tetramerization domain.

In some embodiments, the display polypeptide comprises a type I transmembrane polypeptide, a type II transmembrane polypeptide, a type III transmembrane polypeptide, a GPI-anchored polypeptide, a secreted polypeptide, or a multi-pass transmembrane polypeptide. In some embodiments, the type I transmembrane polypeptide comprises ACE2, TIGIT, or LAG-3. In some embodiments, the type II transmembrane polypeptide comprises OX40-L, TNF-α, or APRIL. In some embodiments, the type III transmembrane polypeptide comprises TACI. In some embodiments, the GPI-anchored polypeptide comprises CD160 or CD48. In some embodiments, the secreted polypeptide comprises IFN-β or LT-α. In some embodiments, the multi-pass transmembrane polypeptide comprises CXCR-4. In some embodiments, the display polypeptide comprises an antibody, a receptor polypeptide, a cytokine, an immune checkpoint polypeptide, a chemokine, or an adhesion factor. In some embodiments, the receptor polypeptide comprises ACE2 or CXCR-4. In some embodiments, the cytokine comprises IFN-β or IFNγ. In some embodiments, the immune checkpoint polypeptide comprises PD-L1 or PD-1. In some embodiments, the chemokine comprises CXCL-12. In some embodiments, the adhesion factor comprises ICAM-1. In some embodiments, the display polypeptide comprises an amino acid sequence that has at least 85% sequence identity to an amino acid sequence according to SEQ ID NOs: 7-26. In some embodiments, the display polypeptide comprises an amino acid sequence that has at least 95% sequence identity to an amino acid sequence according to SEQ ID NOs: 7-26.

In some embodiments, the first recombinant fusion protein and the second recombinant fusion protein have the same transmembrane domains. In some embodiments, the first recombinant fusion protein and the second recombinant fusion protein have different transmembrane domains. In some embodiments, the transmembrane domain anchors the recombinant fusion protein to a bilayer of the enveloped particle. In some embodiments, the transmembrane domain comprises the transmembrane domain of a Vesicular Stomatitis virus glycoprotein (VSV-G). In some embodiments, the transmembrane domain comprises the transmembrane domain and cytosolic domain of a Vesicular Stomatitis virus glycoprotein (VSV-G). In some embodiments, the transmembrane domain comprises the transmembrane domain of a Dengue E protein. In some embodiments, the transmembrane domain comprises the transmembrane domain and cytosolic domain of a Dengue E protein. In some embodiments, the transmembrane domain comprises the transmembrane domain of influenza Neuraminidase (NA). In some embodiments, the transmembrane domain comprises the transmembrane domain and cytosolic domain of influenza Neuraminidase (NA). In some embodiments, the transmembrane domain comprises an amino acid sequence that has at least 85% sequence identity to an amino acid sequence according to SEQ ID NOs: 27-35. In some embodiments, the transmembrane domain comprises an amino acid sequence that has at least 95% sequence identity to an amino acid sequence according to SEQ ID NOs: 27-35.

The present disclosure employs, unless otherwise indicated, conventional molecular biology techniques, which are within the skill of the art. Unless defined otherwise, all technical and scientific terms used herein have the same meaning as is commonly understood by one of ordinary skill in the art.

### Definitions

Throughout this disclosure, various embodiments are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers+/−10% thereof, or 10% below the lower listed limit and 10% above the higher listed limit for the values listed for a range.

The term “coiled coil” as used herein refers to a protein in which 2-7 alpha-helices are coiled together like the strands of a rope. In some embodiments, the coiled coil can be a dimer (FIG. 2 left). In some embodiments, the coiled coil can be a trimer. In some embodiments, the coiled coil can be a tetramer. Coiled coils contain a repeated pattern, hxxhcxc, of hydrophobic (h) and charged (c) amino acid residues, referred to as a heptad repeat. The positions in the heptad repeat are usually labeled abcdefg, where a and d are hydrophobic positions (e.g., isoleucine, leucine, or valine). Folding a sequence with the heptad repeat into an alpha-helical secondary structure causes the hydrophobic residues to be presented as a ‘stripe’ that coils gently around the helix in left-handed fashion, forming an amphipathic structure. The burial of hydrophobic surfaces provide a thermodynamic driving force for the oligomerization. The packing in a coiled-coil interface is tight with almost complete van der Waals contact between the side chains of the a and d residues. The alpha-helices may be parallel or anti-parallel, and usually adopt a left-handed super-coil.

The term “sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. Typically, techniques for determining sequence identity include comparing two nucleotide or amino acid sequences and the determining their percent identity. Sequence comparisons, such as for the purpose of assessing identities, may be performed by any suitable alignment algorithm, including but not limited to the Needleman-Wunsch algorithm (see, e.g., the EMBOSS Needle aligner available at www.ebi.ac.uk/Tools/psa/emboss_needle/, optionally with default settings), the BLAST algorithm (see, e.g., the BLAST alignment tool available at blast.ncbi.nlm.nih.gov/Blast.cgi, optionally with default settings), and the Smith-Waterman algorithm (see, e.g., the EMBOSS Water aligner available at www.ebi.ac.uk/Tools/psa/emboss_water/, optionally with default settings). Optimal alignment may be assessed using any suitable parameters of a chosen algorithm, including default parameters. The “percent identity”, also referred to as “percent homology”, between two sequences may be calculated as the number of exact matches between two optimally aligned sequences divided by the length of the reference sequence and multiplied by 100. Percent identity may also be determined, for example, by comparing sequence information using the advanced BLAST computer program, including version 2.2.9, available from the National Institutes of Health. The BLAST program is based on the alignment method of Karlin and Altschul, Proc. Natl. Acad. Sci. USA 87:2264-2268 (1990) and as discussed in Altschul, et al., J. Mol. Biol. 215:403-410 (1990); Karlin and Altschul, Proc. Natl. Acad. Sci. USA 90:5873-5877 (1993); and Altschul et al., Nucleic Acids Res. 25:3389-3402 (1997). Briefly, the BLAST program defines identity as the number of identical aligned symbols (i.e., nucleotides or amino acids), divided by the total number of symbols in the shorter of the two sequences. The program may be used to determine percent identity over the entire length of the sequences being compared. Default parameters are provided to optimize searches with short query sequences, for example, with the blastp program. The program also allows use of an SEG filter to mask-off segments of the query sequences as determined by the SEG program of Wootton and Federhen, Computers and Chemistry 17:149-163 (1993). High sequence identity generally includes ranges of sequence identity of approximately 80% to 100% and integer values there between.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

The following examples are given for the purpose of illustrating various embodiments of the disclosure and are not meant to limit the present disclosure in any fashion. The present examples, along with the methods described herein are presently representative of preferred embodiments, are exemplary, and are not intended as limitations on the scope of the disclosure. Changes therein and other uses which are encompassed within the spirit of the disclosure as defined by the scope of the claims will occur to those skilled in the art.

### Example 1: Design of MVP Display Vector with L1cc Coiled-Coil Trimeric Domain

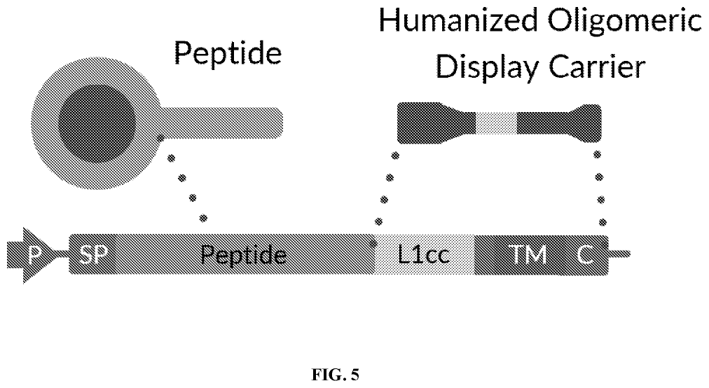

A MVP display vector was designed to present fusion peptides on VLPs or EVs using L1cc as an oligomerization domain. The vector included a mammalian promoter that drove the expression the fusion peptide, which contained a linked signal peptide (SP), displayed peptide, L1cc trimeric peptide, and the transmembrane and cytoplasmic tail domains of VSV-G protein. FIG. 5 illustrates an example design of multivalent particle (MVP) display constructs utilizing the human Line1 coiled-coil (L1cc) trimerization domain, which includes a signal peptide, a display peptide (Peptide), and a humanized oligomeric display carrier comprising L1cc coiled coil, transmembrane domain, and a C-terminal.

Methods: Codon-optimized display peptide sequences were synthesized (Twist) and cloned into specialized display constructs expressing fusion peptides consisting of the displayed protein ectodomain fused to a sequence encoding the desired human oligomerization domain and the transmembrane and cytoplasmic tail domains of VSV-G protein. To generate trimeric humanized MVPs, the ectodomains of displayed peptides were fused to a synthetic sequence encoding the L1cc trimerization domain and the transmembrane and cytoplasmic tail domains of VSV-G.

### Example 2: Production of MVPs Displaying L1cc Fusion Peptides

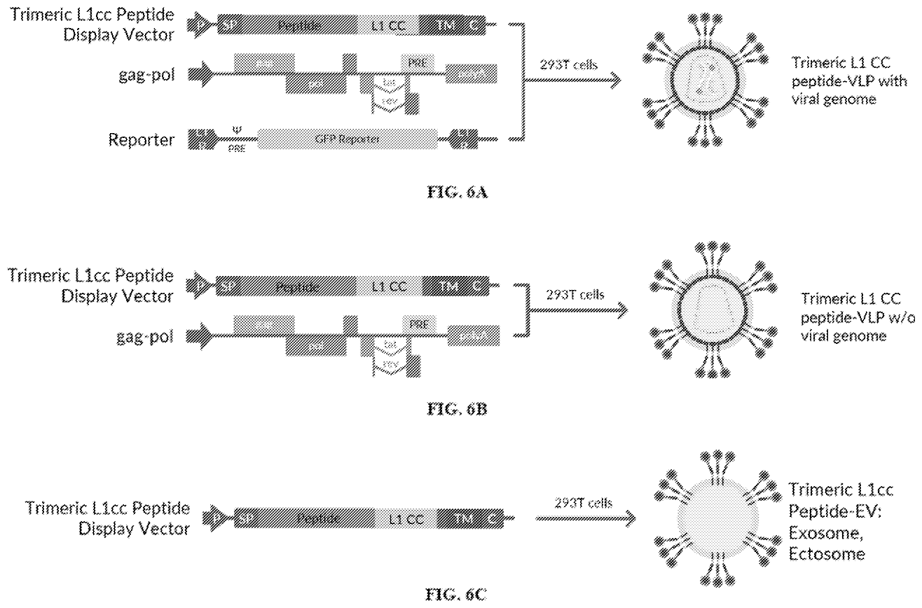

MVPs displaying various types of L1cc fusion peptides were produced as VLPs both with and without genomes and as extracellular vesicles (EVs), such as exosomes and ectosomes. To produce MVPs displaying L1cc fusion peptides as VLPs containing RNA genomes, HEK 293T cells were co-transfected with L1cc display constructs along with a lentiviral packaging construct expressing essential packaging components, including Gag-Pol and Rev proteins, and a viral genome transfer vector encoding a GFP/luciferase reporter (FIG. 6A). To produce MVPs displaying L1cc fusion peptides as VLPs without genomes, HEK 293T cells were co-transfected with humanized display vectors and a lentiviral packaging construct, without a viral genome transfer vector (FIG. 6B). To produce MVPs displaying L1cc fusion peptides as EVs, 293T cells were transfected with humanized peptide display constructs alone (FIG. 6C).

Methods: Humanized MVPs based on VLPs and EVs were produced from transfected HEK 293T cells. To produce lentiviral VLP-based IFN-MVPs with viral genomes, HEK 293T cells were co-transfected with humanized peptide display constructs, along with a lentiviral packaging vector (i.e., psPAX2) and a lentiviral genome transfer vector. To produce lentiviral VLP-based humanized MVPs without viral genomes, HEK 293T cells were co-transfected with humanized peptide display constructs along with a lentiviral packaging vector (i.e., psPAX2). To produce extracellular vesicle-based humanized MVPs, HEK 293T cells were transfected with humanized peptide display constructs alone.

Transfections were prepared by culturing 7.5×10⁶ HEK293T cells (ATCC CRL-3216) overnight in 10-cm dishes (Corning) containing DMEM media with glucose, L-glutamine, and sodium pyruvate (Corning) supplemented with 10% fetal bovine serum (Sigma) and 1% Penicillin Streptomycin (Life Technologies), referred to as “293T Growth Media.” Cells reached around 90% confluence after 24 hours. A transfection DNA mixture was prepared with polyethylenimine (PEI) in OPTI-MEM reduced serum medium (Gibco). The transfection mixture was incubated at room temperature for 15 minutes before being added to the cells, which were then incubated at 37° C. in 5% CO₂. 6 hours post-transfection, the 293T Growth Media was changed to 293T Growth Media supplemented with 0.1% sodium butyrate (referred to as “Transfection Media”) before returning the cells for further incubation. After incubating the cells for 24 hours at 37° C. with 5% CO₂ in the Transfection Media, supernatant containing VLPs or EVs was collected, centrifuged at 1680 rpm for 5 minutes to remove cellular debris, and mixed with 1× polyethylene glycol 8000 solution (PEG, Hampton Research) before being stored at 4° C. for 24 hours to allow for fractionation. Cells were replenished with fresh Transfection Media, and a second particle supernatant collection was performed at 48 hours. Supernatant collections were then pooled, the PEG precipitated, and purified by size exclusion chromatography using Sephacryl™ S-300 High Resolution Beads (Sigma Aldrich).

### Example 3: Characterization of MVPs Displaying L1cc Fusion Peptides

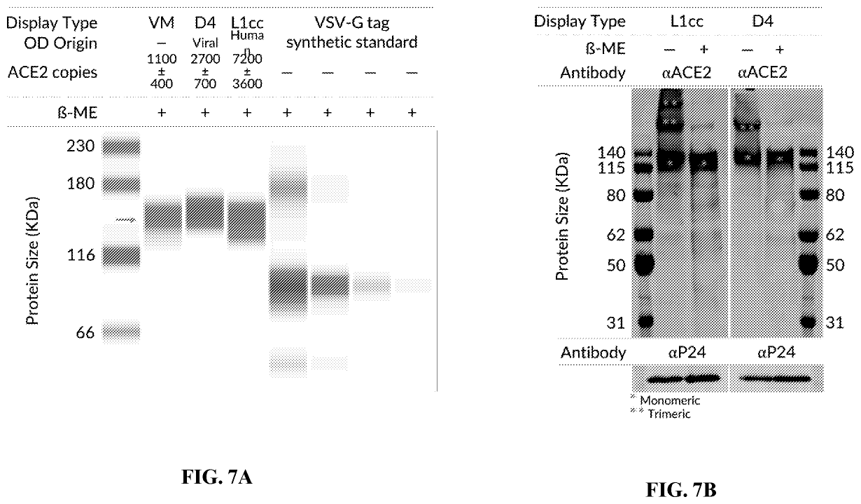

Concentrations of VLP-based and EV-based humanized MVPs were determined via P24 and tunable resistive pulse sensing (TRPS, qNano™), respectively. Based on these concentrations, the copy numbers of displayed humanized L1cc fusion peptides on MVPs were determined by quantitative western blot analysis (FIG. 7A). The protein sizes (KDa) and copy numbers of MVPs with humanized L1cc fusion peptides (L1cc Human) were compared to protein sizes (KDa) of MVPs with VM and D4 viral oligomerization domain. The data show that MVPs with humanized L1cc fusion peptides had a copy number more than 2.5 times that of MVPs with D4 viral oligomerization domain; and more than 7 times that of MVPs with VM. The oligomerization patterns of displayed fusion peptides was determined via non-reducing PAGE analysis (FIG. 7B). The data show that MVPs with humanized L1cc fusion peptides display ACE2 in oligomeric form more efficiently than MVPs using the D4 viral oligomerization domain, as evidenced by more intense bands at larger, oligomeric protein sizes. These results demonstrate that the L1cc domain can be used to more efficiently display oligomeric ACE2 on MVPs.

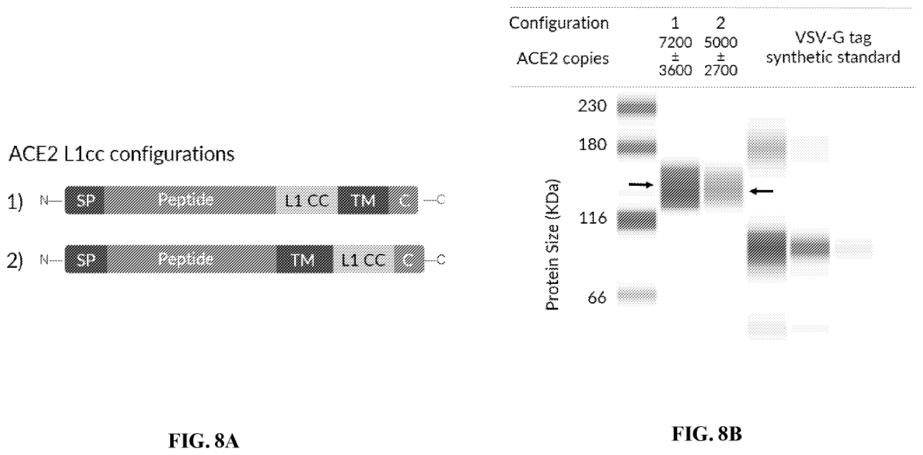

In addition to testing various human oligomerization domains, the configuration of the oligomerization domain on the display peptide was varied to determine the optimal fusion peptide design for achieving efficient oligomerization and maximum MVP copy number. Two variations of L1cc display constructs were produced, with the L1cc domain 1) preceding the VSV-G transmembrane domain or 2) following the VSV-G transmembrane domain (FIG. 8A). FIG. 8B shows that decoy MVPs from both L1cc construct variations displayed functional ACE2 at greater or comparable frequencies to MVPs using VM and D4 display peptides (FIG. 7A). Between the two L1cc variations, having L1cc precede the transmembrane domain (1) yielded MVPs with approximately 2000 more copies of ACE2 per particle than L1cc after the transmembrane domain (2) (FIG. 8B).

Methods: Lentiviral and EV particle quantification by p24 ELISA and Tunable Resistive Pulse Sensing. P24 concentrations in VLP samples of humanized MVPs were determined using an HIV p24 SimpleStep™ ELISA kit (Abcam). Concentrations of lentiviral particles were extrapolated from the assumption that each lentiviral particle contained approximately 2000 molecules of p24, or 1.25×10⁴ pseudovirus particles per picogram of p24 protein.

EV-based humanized MVPs were quantified via tunable resistive pulse sensing (TRPS, qNano™, IZON). Purified EV collections were diluted in 0.2 μm filtered PBS with 0.03% Tween-20™ (Thermo Fisher Scientific) prior to qNano™ analysis. Concentration and size distributions of EV particles were then determined using an NP200 nanopore at a 45.5 mm stretch, and applied voltages between 0.5 and 0.7 V were used to achieve a stable current of 130 nA through the nanopore. Measurements for each EV sample were taken at pressures of 3 mbar, 5 mbar, and 8 mbar. The measurements were considered valid if at least 500 events were recorded, the particle rate was linear, and the root mean squared signal noise was maintained below 10 pA. EV concentrations were then determined by comparison to a standardized multi-pressure calibration using CPC200 (mode diameter: 200 nm) (IZON) carboxylated polystyrene beads diluted 1:200 in 0.2 μM filtered PBS from their original concentration of 7.3×10¹¹ particles per/mL. Measurements were analyzed using IZON Control Suite 3.4 software to determine original sample concentrations.

Methods: Western blot analysis of humanized MVPs. Expression of humanized fusion proteins on humanized MVPs, VLPs, and EVs was confirmed via western blot analysis of purified particles. Samples of purified MVPs were lysed at 4° C. for 10 minutes with cell lysis buffer (Cell Signaling) before being mixed with NuPage™ LDS sample buffer (Thermo Fisher Scientific) and boiled at 95° C. for 5 minutes. Differences in oligomerization were determined by running samples in reducing and non-reducing conditions. Under reducing conditions, 5% 2-Mercaptoethanol (Thermo Fisher Scientific) was added to samples to dissociate oligomerized fusion display proteins on MVPs. Protein samples were then separated on NuPAGE™ 4-12% Bis-Tris gels (Thermo Fisher Scientific) and transferred onto a polyvinylidene fluoride (PVDF) membrane (Life Technologies). PVDF membranes were blocked with TRIS-buffered saline with Tween-20 (TBST) and 5% skim milk (Research Products International) for 1 hour, and then incubated overnight with primary antibody diluted in 5% milk. For fusion display constructs expressing a VSVG-tag, an anti-VSV-G epitope tag rabbit polyclonal antibody (BioLegend, Poly29039) was used at a 1:2000 dilution. The following day, the PVDF membrane was washed 3 times with 1×TBST and stained with a goat-anti-rabbit secondary antibody (IRDye® 680) at a 1:5000 dilution for 60 minutes in 5% milk. Post-secondary antibody staining, the PVDF membrane was again washed 3 times with TBST before imaging on a Licor Odyssey® scanner.

Alternatively, western blot analyses were performed using an automated Simple Western™ size-based protein assay (ProteinSimple). Unless otherwise mentioned, all reagents used were from Protein Simple. Concentrated samples were lysed as described above, before being diluted 1:10 in 0.1× sample buffer for loading on capillaries. Humanized display protein expression levels were identified using the same primary rabbit polyclonal antibody at a 1:400 dilution and an HRP conjugated anti-rabbit secondary antibody (ProteinSimple). Chemiluminescence signal analysis and absolute quantitation were performed using Compass software (ProteinSimple).

Methods: Quantitative western blot analysis of IFN-MVPs. Quantitative western blot analyses were performed to determine the copies of displayed humanized fusion proteins displayed per particle. P24 ELISA or TRPS (qNano™) assays were used to determine MVP sample concentrations. Purified MVP samples were processed and analyzed via western blot under reducing conditions as described above. A synthetic protein standard of known concentration was used to generate a standard curve, from which copy numbers of displayed fusion peptide on respective particles

### Example 4: In Vitro Virus Neutralization by Decoy MVPs Displaying ACE2-L1cc

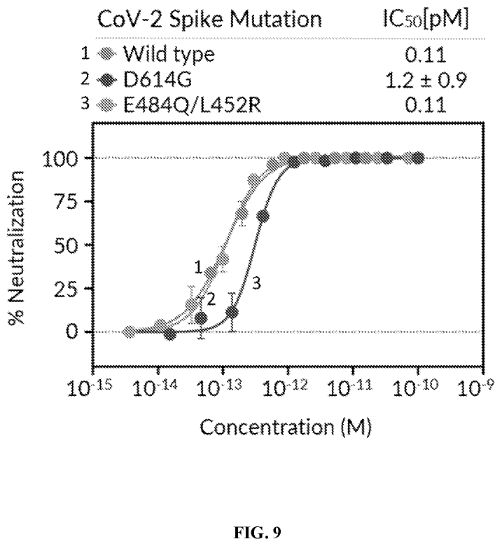

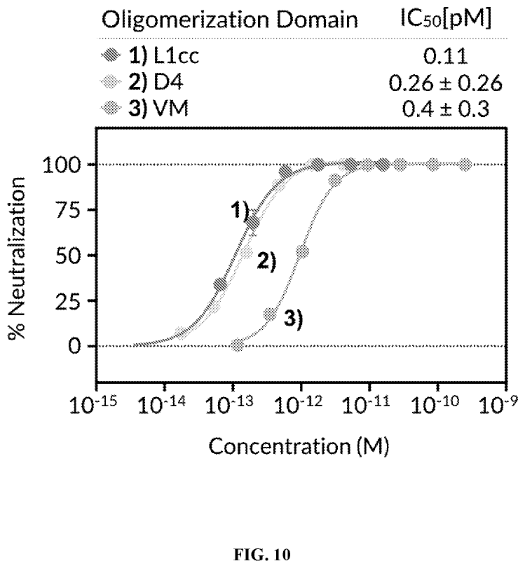

Decoy MVPs displaying ACE2-L1cc were tested in pseudovirus neutralization assays against a panel of SARS COV-2 spike mutations to determine whether the MVPs could retain their neutralizing ability against SARS COV-2. Decoy MVPs displaying trimeric ACE2-L1cc were able to neutralize three different SARS COV-2 spike variants at low or sub-picomolar IC₅₀ in pseudovirus neutralization assays, as shown in FIG. 9. Furthermore, the position of the L1cc trimerization domain did not impact the neutralizing efficacy. ACE2 decoy MVPs derived from both configurations of L1cc display vector neutralized E484Q pseudovirus at sub-picomolar IC₅₀, as shown in FIG. 10.

Methods. H1573/ACE2 target cells were seeded in 96-well, flat-bottom, clear, tissue-culture treated plates (Thermo Fisher Scientific) at 25,000 cells/well with 6 μg/mL polybrene (Sigma) in the corresponding culture medium supplemented with 10% fetal bovine serum (Sigma) and 1% Penicillin Streptomycin (Thermo Fisher). RPMI media with glucose, HEPES Buffer, L-Glutamine, sodium bicarbonate and sodium pyruvate (Gibco) served as base medium for H1573/ACE2 cells. SARS COV-2 pseudovirus was then added to wells at TCID₅₀ concentrations, along with titrated Ab-Antivirus in 9×2-fold or 3-fold serial dilutions, yielding a 10-point dilution curve. Plates containing cells, pseudovirus and humanized ACE2 decoy antivirus were then centrifuged at 800×g, 25° C. for 60 minutes to maximize infection efficiency. 48 hours post-infection, cells were lysed using Firefly Luciferase Lysis Buffer (Biotium) and the resulting lysis was transferred to 96-well, white assay plates (Costar) before luciferase activity was analyzed via GLOMAX™ multi-detection system (Promega). Titrated infection data was then plotted and fitted to a 4-parameter, logistic curve (GraphPad Prism 9.0.0) to calculate the half maximal inhibitory concentration (IC₅₀) of various Ab-Antiviruses neutralizing their respective pseudoviruses.

### Example 5: Decoy MVPs Displaying ACE2-L1cc Rescue hACE2 Transgenic Mice from Lethal SARS CoV-2 Infection

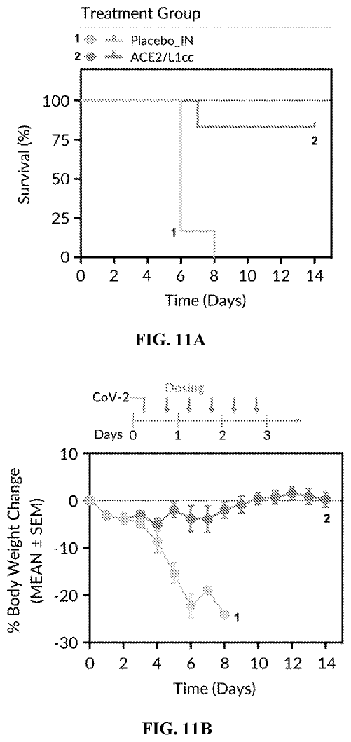

To investigate whether humanized decoy MVPs displaying trimeric ACE2-L1cc could protect ACE2 transgenic mice from lethal SARS COV-2 infection and infection-related symptoms, K18-hACE2 mice were challenged with 2800 PFU of SARS COV-2 (Strain USA-WA1/2020) and treated with 5 doses of decoy MVPs displaying trimeric ACE2-L1cc (1×10¹¹ particles per dose) delivered intranasally (IN). Dosing was initiated 4 hours after infection, and subsequent doses were given twice daily for 2 days following infection. All but one mouse from the treatment group survived infection, while all control mice succumbed around day 6 post-infection (FIG. 11A). Furthermore, treated mice exhibited minimal respiratory distress and transitory weight loss (FIG. 11B). These results demonstrate that the decoy MVPs displaying trimeric ACE2-L1cc rescued K18-hACE2 mice from lethal SARS COV-2 infections and prevented all serious symptoms. The data further demonstrated that decoy MVPs displaying trimeric ACE2-L1cc were highly potent neutralizing agents against live SARS COV-2 viruses in vivo and may be effective as a multivalent therapeutic agent against COVID-19.

Methods. 6 K18-human ACE2 transgenic mice, half male half female, 8-10 weeks old were used in each cohort. Animals were weighed prior to the start of the study. Animals were challenged with 2800 PFU of authentic SARS COV-2 (strain USA-WA1/2020) through IN administration of 50 μL of viral inoculum per nostril. Mice were treated with 5 doses of humanized ACE2 decoy antivirus (1×10¹¹ particles per dose) via IN delivery, beginning 4 hours post-infection and twice a day on day 1 and day 2 post-infection. Animals were monitored twice daily for signs of COVID-19 disease phenotype (ruffled fur, hunched posture, labored breathing) and survival during the study period. Body weights were measured once daily during the study period, and lung tissue was collected and sampled for viral load assays by PRNT. Tissue samples were stored at 80° C. for histology and viral load analysis by qPCR and PRNT analysis.

What is claimed is:

1. A recombinant fusion protein comprising a transmembrane domain, a display polypeptide, and a human oligomerization domain, wherein when the recombinant fusion protein is expressed on a surface of an enveloped particle, the recombinant fusion protein is displayed in an oligomeric format.

2. The recombinant fusion protein of claim 1, wherein the human oligomerization domain comprises a coiled coil motif.

3. The recombinant fusion protein of claim 1 or 2, wherein the human oligomerization domain is a dimerization domain.

4. The recombinant fusion protein of claim 3, wherein the human oligomerization domain comprises a human fibrinogen-like protein 1 coiled coil (hFcc) domain.

5. The recombinant fusion protein of claim 1 or 2, wherein the human oligomerization domain is a trimerization domain.

6. The recombinant fusion protein of claim 5, wherein the human oligomerization domain comprises a LINE-1 retrotransposon orf1p coiled coil (L1cc) domain.

7. The recombinant fusion protein of claim 5, wherein the human oligomerization domain comprises a human lung surfactant protein D coiled coil (hLSPDcc) domain.

8. The recombinant fusion protein of claim 5, wherein the human oligomerization domain comprises a human tetranectin coiled coil (hTETNcc) domain.

9. The recombinant fusion protein of claim 1 or 2, wherein the human oligomerization domain is a tetramerization domain.

10. The recombinant fusion protein of claim 9, wherein the human oligomerization domain comprises a human guanylate-binding protein 1 coiled coil (hGBP1cc) domain.

11. The recombinant fusion protein of any one of claims 1-10, wherein the human oligomerization domain comprises an amino acid sequence that has at least 85% sequence identity to an amino acid sequence according to SEQ ID NOs: 1-6.

12. The recombinant fusion protein of any one of claims 1-10, wherein the human oligomerization domain comprises an amino acid sequence that has at least 95% sequence identity to an amino acid sequence according to SEQ ID NOs: 1-6.

13. The recombinant fusion protein of any one of claims 1-12, wherein when the recombinant fusion protein is expressed on the surface of the enveloped particle, the human oligomerization domain is outside of the enveloped particle.

14. The recombinant fusion protein of any one of claims 1-12, wherein when the recombinant fusion protein is expressed on the surface of the enveloped particle, the human oligomerization domain is outside of the enveloped particle and adjacent to a signal peptide.

15. The recombinant fusion protein of any one of claims 1-12, wherein when the recombinant fusion protein is expressed on the surface of the enveloped particle, the human oligomerization domain is inside of the enveloped particle.

16. The recombinant fusion protein of claim 15, wherein when the recombinant fusion protein is expressed on the surface of the enveloped particle, and wherein the human oligomerization domain is inside of the enveloped particle and adjacent to the transmembrane domain.

17. The recombinant fusion protein of any one of claims 1-16, wherein the recombinant fusion protein further comprises a signal peptide.

18. The recombinant fusion protein of claim 17, wherein domains of the recombinant fusion protein are arranged from the N-terminus to the C-terminus in the following orders:
a) signal peptide, display polypeptide, human oligomerization domain, and transmembrane domain;
b) signal peptide, display polypeptide, transmembrane domain, and human oligomerization domain; or
c) signal peptide, human oligomerization domain, display peptide, and transmembrane domain.

19. The recombinant fusion protein of any one of claims 1-18, wherein the recombinant fusion protein further comprises a cytosolic domain.

20. The recombinant fusion protein of claim 19, wherein domains of the recombinant fusion protein are arranged from the N-terminus to the C-terminus in the following orders:
a) signal peptide, display polypeptide, human oligomerization domain, transmembrane domain, and cytosolic domain;
b) signal peptide, display polypeptide, transmembrane domain, human oligomerization domain, and cytosolic domain; or
c) signal peptide, human oligomerization domain, display peptide, transmembrane domain, and cytosolic domain.

21. The recombinant fusion protein of any one of claims 1-20, wherein the enveloped particle comprises an enveloped viral-like particle, an enveloped virus, or an extracellular vesicle.

22. The recombinant fusion protein of claim 21, wherein the extracellular vesicle comprises an exosome.

23. The recombinant fusion protein of claim 21, wherein the extracellular vesicle comprises an ectosome.

24. The recombinant fusion protein of any one of claims 1-23, wherein the display polypeptide targets a cell surface protein that is oligomerized on a target cell for agonistic function.

25. The recombinant fusion protein of any one of claims 1-23, wherein the display polypeptide targets a cell surface protein that is oligomerized on a target cell for antagonistic function.

26. The recombinant fusion protein of claim 24 or 25, wherein the cell surface protein is dimerized, and the human oligomerization domain is a dimerization domain.

27. The recombinant fusion protein of claim 24 or 25, wherein the cell surface protein is trimerized, and the human oligomerization domain is a trimerization domain.

28. The recombinant fusion protein of claim 24 or 25, wherein the cell surface protein is trimerized, and the human oligomerization domain is a tetramerization domain.

29. The recombinant fusion protein of any one of claims 1-28, wherein the display polypeptide comprises a type I transmembrane polypeptide, a type II transmembrane polypeptide, a type III transmembrane polypeptide, a GPI-anchored polypeptide, a secreted polypeptide, or a multi-pass transmembrane polypeptide.

30. The recombinant fusion protein of claim 29, wherein the type I transmembrane polypeptide comprises ACE2, TIGIT, or LAG-3.

31. The recombinant fusion protein of claim 29, wherein the type II transmembrane polypeptide comprises OX40-L, TNF-α, or APRIL.

32. The recombinant fusion protein of claim 29, wherein the type III transmembrane polypeptide comprises TACI.

33. The recombinant fusion protein of claim 29, wherein the GPI-anchored polypeptide comprises CD160 or CD48.

34. The recombinant fusion protein of claim 29, wherein the secreted polypeptide comprises IFN-β or LT-α.

35. The recombinant fusion protein of claim 29, wherein the multi-pass transmembrane polypeptide comprises CXCR-4.

36. The recombinant fusion protein of any one of claims 1-28, wherein the display polypeptide comprises an antibody, a receptor polypeptide, a cytokine, an immune checkpoint polypeptide, a chemokine, or an adhesion factor.

37. The recombinant fusion protein of claim 36, wherein the receptor polypeptide comprises ACE2 or CXCR-4.

38. The recombinant fusion protein of claim 36, wherein the cytokine comprises IFN-β or IFNγ.

39. The recombinant fusion protein of claim 36, wherein the immune checkpoint polypeptide comprises PD-L1 or PD-1.

40. The recombinant fusion protein of claim 36, wherein the chemokine comprises CXCL-12.

41. The recombinant fusion protein of claim 36, wherein the adhesion factor comprises ICAM-1.

42. The recombinant fusion protein of any one of claims 1-41, wherein the display polypeptide comprises an amino acid sequence that has at least 85% sequence identity to an amino acid sequence according to SEQ ID NOs: 7-26.

43. The recombinant fusion protein of any one of claims 1-41, wherein the display polypeptide comprises an amino acid sequence that has at least 95% sequence identity to an amino acid sequence according to SEQ ID NOs: 7-26.

44. The recombinant fusion protein of any one of claims 1-42, wherein the transmembrane domain anchors the recombinant fusion protein to a bilayer of the enveloped particle.

45. The recombinant fusion protein of any one of claims 1-44, wherein the transmembrane domain comprises the transmembrane domain of a Vesicular Stomatitis virus glycoprotein (VSV-G).

46. The recombinant fusion protein of claim 45, wherein the recombinant fusion protein comprises the transmembrane domain and cytosolic domain of a Vesicular Stomatitis virus glycoprotein (VSV-G).

47. The recombinant fusion protein of any one of claims 1-44, wherein the transmembrane domain comprises the transmembrane domain of a Dengue E protein.

48. The recombinant fusion protein of claim 47, wherein the recombinant fusion protein comprises the transmembrane domain and cytosolic domain of a Dengue E protein.

49. The recombinant fusion protein of any one of claims 1-44, wherein the transmembrane domain comprises the transmembrane domain of influenza Neuraminidase (NA).

50. The recombinant fusion protein of claim 49, wherein the recombinant fusion protein comprises the transmembrane domain and cytosolic domain of influenza Neuraminidase (NA).

51. The recombinant fusion protein of any one of claims 1-50, wherein the transmembrane domain comprises an amino acid sequence that has at least 85% sequence identity to an amino acid sequence according to SEQ ID NOs: 27-35.

52. The recombinant fusion protein of any one of claims 1-50, wherein the transmembrane domain comprises an amino acid sequence that has at least 95% sequence identity to an amino acid sequence according to SEQ ID NOs: 27-35.

53. The recombinant fusion protein of any one of claims 1-51, wherein when the recombinant fusion protein is expressed on the surface of the enveloped particle, the recombinant fusion protein is expressed at a valency of about 10 copies on the surface of the enveloped particle.

54. The recombinant fusion protein of any one of claims 1-51, wherein when the recombinant fusion protein is expressed on the surface of the enveloped particle, the recombinant fusion protein is expressed at a valency of about 10 to 15 copies on the surface of the enveloped particle.

55. The recombinant fusion protein of any one of claims 1-51, wherein when the recombinant fusion protein is expressed on the surface of the enveloped particle, the recombinant fusion protein is expressed at a valency of at least about 25 copies on the surface of the enveloped particle.

56. The recombinant fusion protein of any one of claims 1-51, wherein when the recombinant fusion protein is expressed on the surface of the enveloped particle, the recombinant fusion protein is expressed at a valency of at least about 50 copies on the surface of the enveloped particle.

57. The recombinant fusion protein of any one of claims 1-51, wherein when the recombinant fusion protein is expressed on the surface of the enveloped particle, the recombinant fusion protein is expressed at a valency of at least about 100 copies on the surface of the enveloped particle.

58. The recombinant fusion protein of any one of claims 1-51, wherein when the recombinant fusion protein is expressed on the surface of the enveloped particle, the recombinant fusion protein is expressed at a valency of at least about 200 copies on the surface of the enveloped particle.

59. The recombinant fusion protein of any one of claims 1-51, wherein when the recombinant fusion protein is expressed on the surface of the enveloped particle, the recombinant fusion protein is expressed at a valency of at least about 400 copies on the surface of the enveloped particle.

60. The recombinant fusion protein of any one of claims 1-51, wherein when the recombinant fusion protein is expressed on the surface of the enveloped particle, the recombinant fusion protein is expressed at a valency of at least about 600 copies on the surface of the enveloped particle.

61. The recombinant fusion protein of any one of claims 1-51, wherein when the recombinant fusion protein is expressed on the surface of the enveloped particle, the recombinant fusion protein is expressed at least about 1000 copies on the surface of the enveloped particle.

62. The recombinant fusion protein of any one of claims 1-51, wherein when the recombinant fusion protein is expressed on the surface of the enveloped particle, the recombinant fusion protein is expressed at least about 2000 copies on the surface of the enveloped particle.

63. An enveloped particle comprising the recombinant fusion protein according to any one of claims 1-62.

64. An enveloped particle comprising:
a) a first recombinant fusion protein that is displayed in an oligomeric format on the surface of the enveloped particle wherein the first recombinant fusion protein comprises a transmembrane domain, a display polypeptide, and a human oligomerization domain; and
b) a second recombinant fusion protein that is displayed in a monomeric format on the surface of the enveloped particle wherein the second recombinant fusion protein comprises a transmembrane domain and a display polypeptide.

65. The enveloped particle of claim 64, wherein the human oligomerization domain comprises a human coiled coil motif.

66. The enveloped particle of claim 64, wherein the human oligomerization domain is a dimerization domain.

67. The enveloped particle of claim 66, wherein the human oligomerization domain comprises a human fibrinogen-like protein 1 coiled coil (hFcc) domain.

68. The enveloped particle of claim 64, wherein the human oligomerization domain is a trimerization domain.

69. The enveloped particle of claim 68, wherein the human oligomerization domain comprises a LINE-1 retrotransposon orf1p coiled coil (L1cc) domain.

70. The enveloped particle of claim 68, wherein the human oligomerization domain comprises a human lung surfactant protein D coiled coil (hLSPDcc) domain.

71. The enveloped particle of claim 68, wherein the human oligomerization domain comprises a human tetranectin coiled coil (hTETNcc) domain.

72. The enveloped particle of claim 64, wherein the human oligomerization domain is a tetramerization domain.

73. The enveloped particle of claim 72, wherein the human oligomerization domain comprises a human guanylate-binding protein 1 coiled coil (hGBP1cc) domain.

74. The enveloped particle of any one of claims 65-72, wherein the human oligomerization domain comprises an amino acid sequence that has at least 85% sequence identity to an amino acid sequence according to SEQ ID NOs: 1-6.

75. The enveloped particle of any one of claims 65-74, wherein the human oligomerization domain comprises an amino acid sequence that has at least 95% sequence identity to an amino acid sequence according to SEQ ID NOs: 1-6.

76. The enveloped particle of any one of claims 64-74, wherein when the first recombinant fusion protein is expressed on the surface of the enveloped particle, the human oligomerization domain is outside of the enveloped particle.

77. The enveloped particle of claim 76, wherein when the first recombinant fusion protein is expressed on the surface of the enveloped particle, the human oligomerization domain is outside of the enveloped particle and adjacent to a signal peptide.

78. The enveloped particle of any one of claims 64-74, wherein when the first recombinant fusion protein is expressed on the surface of the enveloped particle, the human oligomerization domain is inside of the enveloped particle.

79. The enveloped particle of claim 78, wherein when the first recombinant fusion protein is expressed on the surface of the enveloped particle, the human oligomerization domain is inside of the enveloped particle and adjacent to the transmembrane domain.

80. The enveloped particle of any one of claims 64-79, wherein the first recombinant fusion protein further comprises a signal peptide.

81. The enveloped particle of claim 80, wherein domains of the first recombinant fusion protein are arranged from the N-terminus to the C-terminus in the following orders:
a) signal peptide, display polypeptide, human oligomerization domain, and transmembrane domain;
b) signal peptide, display polypeptide, transmembrane domain, and human oligomerization domain; or
c) signal peptide, human oligomerization domain, display peptide, and transmembrane domain.

82. The enveloped particle of any one of claims 64-81, wherein the first recombinant fusion protein further comprises a cytosolic domain.

83. The enveloped particle of claim 82, wherein domains of the first recombinant fusion protein are arranged from the N-terminus to the C-terminus in the following orders:
a) signal peptide, display polypeptide, human oligomerization domain, transmembrane domain, and cytosolic domain;
b) signal peptide, display polypeptide, transmembrane domain, human oligomerization domain, and cytosolic domain; or
c) signal peptide, human oligomerization domain, display peptide, transmembrane domain, and cytosolic domain.

84. The enveloped particle of any one of claims 64-83, wherein the enveloped particle comprises an enveloped viral-like particle, an enveloped virus, an extracellular vesicle.

85. The enveloped particle of claim 84, wherein the extracellular vesicle comprises an exosome.

86. The enveloped particle of claim 84, wherein the extracellular vesicle comprises an ectosome.

87. The enveloped particle of any one of claims 64-86, wherein the first recombinant fusion protein and the second recombinant fusion protein have the same display polypeptide.

88. The enveloped particle of any one of claims 64-86, wherein the first recombinant fusion protein and the second recombinant fusion protein have different display polypeptides.

89. The enveloped particle of any one of claims 64-88, wherein the display polypeptide targets a cell surface protein that is oligomerized on a target cell for agonistic function.

90. The enveloped particle of any one of claims 64-88, wherein the display polypeptide targets a cell surface protein that is oligomerized on a target cell for antagonistic function.

91. The enveloped particle of claim 89 or 90, wherein the cell surface protein is dimerized and the human oligomerization domain is a dimerization domain.

92. The enveloped particle of claim 89 or 90, wherein the cell surface protein is trimerized and the human oligomerization domain is a trimerization domain.

93. The enveloped particle of claim 89 or 90, wherein the cell surface protein is tetramerized and the human oligomerization domain is a tetramerization domain.

94. The enveloped particle of any one of claims 64-93, wherein the display polypeptide comprises a type I transmembrane polypeptide, a type II transmembrane polypeptide, a type III transmembrane polypeptide, a GPI-anchored polypeptide, a secreted polypeptide, or a multi-pass transmembrane polypeptide.

95. The enveloped particle of claim 94, wherein the type I transmembrane polypeptide comprises ACE2, TIGIT, or LAG-3.

96. The enveloped particle of claim 94, wherein the type II transmembrane polypeptide comprises OX40-L, TNF-α, or APRIL.

97. The enveloped particle of claim 94, wherein the type III transmembrane polypeptide comprises TACI.

98. The enveloped particle of claim 94, wherein the GPI-anchored polypeptide comprises CD160 or CD48.

99. The enveloped particle of claim 94, wherein the secreted polypeptide comprises IFN-β or LT-α.

100. The enveloped particle of claim 94, wherein the multi-pass transmembrane polypeptide comprises CXCR-4.

101. The enveloped particle of any one of claims 64-93, wherein the display polypeptide comprises an antibody, a receptor polypeptide, a cytokine, an immune checkpoint polypeptide, a chemokine, or an adhesion factor.

102. The enveloped particle of claim 101, wherein the receptor polypeptide comprises ACE2 or CXCR-4.

103. The enveloped particle of claim 101, wherein the cytokine comprises IFN-β or IFNγ.

104. The enveloped particle of claim 101, wherein the immune checkpoint polypeptide comprises PD-L1 or PD-1.

105. The enveloped particle of claim 101, wherein the chemokine comprises CXCL-12.

106. The enveloped particle of claim 101, wherein the adhesion factor comprises ICAM-1.

107. The enveloped particle of claim 101, wherein the display polypeptide comprises an amino acid sequence that has at least 85% sequence identity to an amino acid sequence according to SEQ ID NOs: 7-26.

108. The enveloped particle of claim 101, wherein the display polypeptide comprises an amino acid sequence that has at least 95% sequence identity to an amino acid sequence according to SEQ ID NOs: 7-26.

109. The enveloped particle of any one of claims 64-107, wherein the first recombinant fusion protein and the second recombinant fusion protein have the same transmembrane domains.

110. The enveloped particle of any one of claims 64-107, wherein the first recombinant fusion protein and the second recombinant fusion protein have different transmembrane domains.

111. The enveloped particle of any one of claims 64-110, wherein the transmembrane domain anchors the recombinant fusion protein to a bilayer of the enveloped particle.

112. The enveloped particle of any one of claims 64-111, wherein the transmembrane domain comprises the transmembrane domain of a Vesicular Stomatitis virus glycoprotein (VSV-G).

113. The enveloped particle of claim 112, wherein the first recombinant fusion protein comprises the transmembrane domain and cytosolic domain of a Vesicular Stomatitis virus glycoprotein (VSV-G).

114. The enveloped particle of any one of claims 64-111, wherein the transmembrane domain comprises the transmembrane domain of a Dengue E protein.

115. The enveloped particle of claim 114, wherein the first recombinant fusion protein comprises the transmembrane domain and cytosolic domain of a Dengue E protein.

116. The enveloped particle of any one of claims 64-111, wherein the transmembrane domain comprises the transmembrane domain of influenza Neuraminidase (NA).

117. The enveloped particle of claim 116, wherein the first recombinant fusion protein comprises the transmembrane domain and cytosolic domain of influenza Neuraminidase (NA).

118. The enveloped particle of any one of claims 64-117, wherein the transmembrane domain comprises an amino acid sequence that has at least 85% sequence identity to an amino acid sequence according to SEQ ID NOs: 27-35.

119. The enveloped particle of any one of claims 64-117, wherein the transmembrane domain comprises an amino acid sequence that has at least 95% sequence identity to an amino acid sequence according to SEQ ID NOs: 27-35.

120. The enveloped particle of any one of claims 64-118, wherein when the first recombinant fusion protein is expressed on the surface of the enveloped particle the first recombinant fusion protein is expressed at a valency of about 10 copies on the surface of the enveloped particle.

121. The enveloped particle of any one of claims 64-118, wherein when the first recombinant fusion protein is expressed on the surface of the enveloped particle the first recombinant fusion protein is expressed at a valency of about 10 to 15 copies on the surface of the enveloped particle.

122. The enveloped particle of any one of claims 64-118, wherein when the first recombinant fusion protein is expressed on the surface of the enveloped particle the first recombinant fusion protein is expressed at a valency of at least about 25 copies on the surface of the enveloped particle.

123. The enveloped particle of any one of claims 64-118, wherein when the first recombinant fusion protein is expressed on the surface of the enveloped particle the first recombinant fusion protein is expressed at a valency of at least about 50 copies on the surface of the enveloped particle.

124. The enveloped particle of any one of claims 64-118, wherein when the first recombinant fusion protein is expressed on the surface of the enveloped particle the first recombinant fusion protein is expressed at a valency of at least about 100 copies on the surface of the enveloped particle.

125. The enveloped particle of any one of claims 64-118, wherein when the first recombinant fusion protein is expressed on the surface of the enveloped particle the first recombinant fusion protein is expressed at a valency of at least about 200 copies on the surface of the enveloped particle.

126. The enveloped particle of any one of claims 64-118, wherein when the first recombinant fusion protein is expressed on the surface of the enveloped particle the first recombinant fusion protein is expressed at a valency of at least about 400 copies on the surface of the enveloped particle.

127. The enveloped particle of any one of claims 64-118, wherein when the first recombinant fusion protein is expressed on the surface of the enveloped particle the first recombinant fusion protein is expressed at a valency of at least about 600 copies on the surface of the enveloped particle.

128. The enveloped particle of any one of claims 64-118, wherein when the first recombinant fusion protein is expressed on the surface of the enveloped particle the first recombinant fusion protein is expressed on the surface of the enveloped particle at a valency of at least about 1000 copies on the surface of the enveloped particle.

129. The enveloped particle of any one of claims 64-118, wherein when the second recombinant fusion protein is expressed on the surface of the enveloped particle the second recombinant fusion protein is expressed at a valency of about 10 copies on the surface of the enveloped particle.

130. The enveloped particle of any one of claims 64-118, wherein when the second recombinant fusion protein is expressed on the surface of the enveloped particle the second recombinant fusion protein is expressed at a valency of about 10 to 15 copies on the surface of the enveloped particle.

131. The enveloped particle of any one of claims 64-118, wherein when the second recombinant fusion protein is expressed on the surface of the enveloped particle the second recombinant fusion protein is expressed at a valency of at least about 25 copies on the surface of the enveloped particle.

132. The enveloped particle of any one of claims 64-118, wherein when the second recombinant fusion protein is expressed on the surface of the enveloped particle the second recombinant fusion protein is expressed at a valency of at least about 50 copies on the surface of the enveloped particle.

133. The enveloped particle of any one of claims 64-118, wherein when the second recombinant fusion protein is expressed on the surface of the enveloped particle the second recombinant fusion protein is expressed at a valency of at least about 100 copies on the surface of the enveloped particle.

134. The enveloped particle of any one of claims 64-118, wherein when the second recombinant fusion protein is expressed on the surface of the enveloped particle the second recombinant fusion protein is expressed at a valency of at least about 200 copies on the surface of the enveloped particle.

135. The enveloped particle of any one of claims 64-118, wherein when the second recombinant fusion protein is expressed on the surface of the enveloped particle the second recombinant fusion protein is expressed at a valency of at least about 400 copies on the surface of the enveloped particle.

136. The enveloped particle of any one of claims 64-118, wherein when the second recombinant fusion protein is expressed on the surface of the enveloped particle the second recombinant fusion protein is expressed at a valency of at least about 600 copies on the surface of the enveloped particle.

137. The enveloped particle of any one of claims 64-118, wherein when the second recombinant fusion protein is expressed on the surface of the enveloped particle the second recombinant fusion protein is expressed on the surface of the enveloped particle at a valency of at least about 1000 copies on the surface of the enveloped particle.

138. The enveloped particle of any one of claims 64-118, wherein when the second recombinant fusion protein is expressed on the surface of the enveloped particle the second recombinant fusion protein is expressed on the surface of the enveloped particle at a valency of at least about 2000 copies on the surface of the enveloped particle.

139. A composition comprising a nucleic acid sequence that encodes the recombinant fusion protein of any one of claims 1-62.

140. A composition comprising a nucleic acid sequence that encodes the first recombinant fusion protein of any one of claims 64-118 and the second recombinant fusion protein of any one of claims 64-118.

141. The composition of claim 139 or 140, wherein the composition further comprises a second nucleic acid sequence that encodes one or more packaging viral proteins.

142. The composition of claim 141, wherein the one or more packaging viral proteins is a lentiviral protein, a retroviral protein, an adenoviral protein, or combinations thereof.

143. The composition of claim 141, wherein the one or more packaging viral proteins comprises gag, pol, pre, tat, rev, or combinations thereof.

144. The composition of any one of claims 139-143, further comprising a third nucleic acid sequence that encodes a reporter, a therapeutic molecule, or combinations thereof.

145. The composition of claim 144, wherein the reporter is a fluorescent protein.

146. The composition of claim 144, wherein the reporter is a luciferase.

147. The composition of claim 144, wherein the fluorescent protein is green fluorescent protein.

148. The composition of claim 144, wherein the therapeutic molecule is an immune modulating protein, a cellular signal modulating molecule, a proliferation modulating molecule, a cell death modulating molecule, or combinations thereof.

149. The composition of any one of claims 139-148, wherein the nucleic acid sequence that encodes the recombinant fusion protein and the second nucleic acid sequence and the third nucleic acid sequence are within a same vector.

150. The composition of any one of claims 139-148, wherein the nucleic acid sequence that encodes the first recombinant fusion protein and the second recombinant fusion protein and the second nucleic acid sequence and the third nucleic acid sequence are within a same vector.

151. The composition of any one of claims 139-148, wherein the nucleic acid sequence that encodes the recombinant fusion protein and the second nucleic acid sequence and the third nucleic acid sequence are within different vectors.

152. The composition of any one of claims 139-148, wherein the nucleic acid sequence that encodes the first recombinant fusion protein and the second recombinant fusion protein and the second nucleic acid sequence and the third nucleic acid sequence are within different vectors.

153. The composition of any one of claims 149-152, wherein the vector is a lentivirus vector, an adenovirus vector, or an adeno-associated virus vector.

154. A method of agonizing or antagonizing a cell that has an oligomerized cell surface protein comprising contacting the cell with an enveloped particle comprising a first recombinant fusion protein that is displayed in an oligomeric format on the surface of the enveloped particle, wherein the first recombinant fusion protein comprises a transmembrane domain, a display polypeptide, and a human oligomerization domain.

155. The method of claim 154, wherein the enveloped particle further comprises a second recombinant fusion protein that is displayed in a monomeric format on the surface of the enveloped particle, wherein the second recombinant fusion protein comprises a transmembrane domain and a display polypeptide.

156. The method of claim 154 or 155, wherein the human oligomerization domain comprises a human coiled coil motif.

157. The method of claim 154, wherein the human oligomerization domain is a dimerization domain.

158. The method of claim 157, wherein the human oligomerization domain comprises a human fibrinogen-like protein 1 coiled coil (hFcc) domain.

159. The method of claim 154, wherein the human oligomerization domain is a trimerization domain.

160. The method of claim 159, wherein the human oligomerization domain comprises a LINE-1 retrotransposon orf1p coiled coil (L1cc) domain.

161. The method of claim 159, wherein the human oligomerization domain comprises a human lung surfactant protein D coiled coil (hLSPDcc) domain.

162. The method of claim 159, wherein the human oligomerization domain comprises a human tetranectin coiled coil (hTETNcc) domain.

163. The method of claim 154, wherein the human oligomerization domain is a tetramerization domain.

164. The method of claim 163, wherein the human oligomerization domain comprises a human guanylate-binding protein 1 coiled coil (hGBP1cc) domain.

165. The method of any one of claims 154-164, wherein the human oligomerization domain comprises an amino acid sequence that has at least 85% sequence identity to an amino acid sequence according to SEQ ID NOs: 1-6.

166. The method of any one of claims 154-164, wherein the human oligomerization domain comprises an amino acid sequence that has at least 95% sequence identity to an amino acid sequence according to SEQ ID NOs: 1-6.

167. The method of any one of claims 154-166, wherein when the first recombinant fusion protein is expressed on the surface of the enveloped particle, the human oligomerization domain is outside of the enveloped particle.

168. The method of claim 167, wherein when the first recombinant fusion protein is expressed on the surface of the enveloped particle, the human oligomerization domain is outside of the enveloped particle and adjacent to a signal peptide.

169. The method of any one of claims 154-166, wherein when the first recombinant fusion protein is expressed on the surface of the enveloped particle, the human oligomerization domain is inside of the enveloped particle.

170. The method of claim 169, wherein when the first recombinant fusion protein is expressed on the surface of the enveloped particle, the human oligomerization domain is inside of the enveloped particle and adjacent to the transmembrane domain.

171. The method of any one of claims 154-170, wherein the first recombinant fusion protein comprises a signal peptide.

172. The method of any one of claims 154-171, wherein domains of the first recombinant fusion protein are arranged from the N-terminus to the C-terminus in the following orders:
a) signal peptide, display polypeptide, human oligomerization domain, and transmembrane domain;
b) signal peptide, display polypeptide, transmembrane domain, and human oligomerization domain; or
c) signal peptide, human oligomerization domain, display peptide, and transmembrane domain.

173. The method of claim 172, wherein the first recombinant fusion protein further comprises a cytosolic domain.

174. The method of any one of claims 154-171, wherein domains of the first recombinant fusion protein are arranged from the N-terminus to the C-terminus in the following orders:
a) signal peptide, display polypeptide, human oligomerization domain, transmembrane domain, and cytosolic domain;
b) signal peptide, display polypeptide, transmembrane domain, human oligomerization domain, and cytosolic domain; or
c) signal peptide, human oligomerization domain, display peptide, transmembrane domain, and cytosolic domain.

175. The method of claim 172, wherein the enveloped particle comprises an enveloped viral-like particle, an enveloped virus, an extracellular vesicle.

176. The method of claim 175, wherein the extracellular vesicle comprises an exosome.

177. The method of claim 175, wherein the extracellular vesicle comprises an ectosome.

178. The method of any one of claims 154-177, wherein the first recombinant fusion protein and the second recombinant fusion protein have the same display polypeptide.

179. The method of any one of claims 154-177, wherein the first recombinant fusion protein and the second recombinant fusion protein have different display polypeptides.

180. The method of any one of claims 154-179, wherein the display polypeptide targets the cell surface protein that is oligomerized on a target cell for agonistic function.

181. The method of any one of claims 154-179, wherein the display polypeptide targets the cell surface protein that is oligomerized on a target cell for antagonistic function.

182. The method of claim 180 or 181, wherein the cell surface protein is dimerized and the coiled coil oligomerization domain is a dimerization domain.

183. The method of claim 180 or 181, wherein the cell surface protein is trimerized and the coiled coil oligomerization domain is a trimerization domain.

184. The method of claim 180 or 181, wherein the cell surface protein is tetramerized and the coiled coil oligomerization domain is a tetramerization domain.

185. The method of any one of claims 154-184, wherein the display polypeptide comprises a type I transmembrane polypeptide, a type II transmembrane polypeptide, a type III transmembrane polypeptide, a GPI-anchored polypeptide, a secreted polypeptide, or a multi-pass transmembrane polypeptide.

186. The method of claim 185, wherein the type I transmembrane polypeptide comprises ACE2, TIGIT, or LAG-3.

187. The method of claim 185, wherein the type II transmembrane polypeptide comprises OX40-L, TNF-α, or APRIL.

188. The method of claim 185, wherein the type III transmembrane polypeptide comprises TACI.

189. The method of claim 185, wherein the GPI-anchored polypeptide comprises CD160 or CD48.

190. The method of claim 185, wherein the secreted polypeptide comprises IFN-β or LT-α.

191. The method of claim 185, wherein the multi-pass transmembrane polypeptide comprises CXCR-4.

192. The method of any one of claims 154-184, wherein the display polypeptide comprises an antibody, a receptor polypeptide, a cytokine, an immune checkpoint polypeptide, a chemokine, or an adhesion factor.

193. The method of claim 192, wherein the receptor polypeptide comprises ACE2 or CXCR-4.

194. The method of claim 192, wherein the cytokine comprises IFN-β or IFNγ.

195. The method of claim 192, wherein the immune checkpoint polypeptide comprises PD-L1 or PD-1.

196. The method of claim 192, wherein the chemokine comprises CXCL-12.

197. The method of claim 192, wherein the adhesion factor comprises ICAM-1.

198. The method of any one of claims 153-196, wherein the display polypeptide comprises an amino acid sequence that has at least 85% sequence identity to an amino acid sequence according to SEQ ID NOs: 7-26.

199. The method of any one of claims 153-196, wherein the display polypeptide comprises an amino acid sequence that has at least 95% sequence identity to an amino acid sequence according to SEQ ID NOs: 7-26.

200. The method of any one of claims 154-198, wherein the first recombinant fusion protein and the second recombinant fusion protein have the same transmembrane domains.

201. The method of any one of claims 154-198, wherein the first recombinant fusion protein and the second recombinant fusion protein have different transmembrane domains.

202. The method of any one of claims 154-201, wherein the transmembrane domain anchors the recombinant fusion protein to a bilayer of the enveloped particle.

203. The method of any one of claims 154-202, wherein the transmembrane domain comprises the transmembrane domain of a Vesicular Stomatitis virus glycoprotein (VSV-G).

204. The method of claim 203, wherein the transmembrane domain comprises the transmembrane domain and cytosolic domain of a Vesicular Stomatitis virus glycoprotein (VSV-G).

205. The method of any one of claims 154-202, wherein the transmembrane domain comprises the transmembrane domain of a Dengue E protein.

206. The method of claim 205, wherein the transmembrane domain comprises the transmembrane domain and cytosolic domain of a Dengue E protein.

207. The method of any one of claims 154-202, wherein the transmembrane domain comprises the transmembrane domain of influenza Neuraminidase (NA).

208. The method of claim 207, wherein the transmembrane domain comprises the transmembrane domain and cytosolic domain of influenza Neuraminidase (NA).

209. The method of any one of claims 154-208, wherein the transmembrane domain comprises an amino acid sequence that has at least 85% sequence identity to an amino acid sequence according to SEQ ID NOs: 27-35.

210. The method of any one of claims 154-209, wherein the transmembrane domain comprises an amino acid sequence that has at least 95% sequence identity to an amino acid sequence according to SEQ ID NOs: 27-35.

211. A composition comprising a multivalent particle (MVP), wherein the MVP comprises an enveloped particle that displays at least about 10 copies of a peptide on a surface of the MVP, wherein at least one surface protein on the MVP comprises an oligomerized format, and wherein the peptide forms a multivalent interaction with a cognate ligand on a target cell or a target virus.

212. A method of using a multivalent particle (MVP) displaying an oligomerized peptide as immunogen to generate protective immunity against a target antigen, wherein the target antigen is a bacterial antigen, a viral antigen, a fungal antigen, or a tumor antigen, wherein the target antigen is displayed at least about 10 copies of a peptide on a surface of the MVP, and wherein at least one surface protein on the MVP comprises an oligomerized format.

213. A method of using a multivalent particle (MVP) displaying oligomerized peptide as immunogen to generate protective immunity against a target antigen, wherein the target antigen is a bacterial antigen, a viral antigen, a fungal antigen, or a tumor antigen, wherein the MVP comprises a double stranded ribonucleic viral genome as a toll-like receptor agonist adjuvant.


SEQUENCE LISTING (35 sequences)

SEQ ID NO: 1 - protein, 105 aa, Homo sapiens
RSNYSELREDIQTKGKEVENFEKNLEECITRITNTEKCLKELMELKTKARELREECRSLRSRCDQLEERVSAMEDEMNEMKREGKFREKRIKRNEQSLQEIWDYV

SEQ ID NO: 2 - protein, 54 aa, Homo sapiens
NLVTAFSNMDDMLQKAHLVIEGTFIYLRDSTEFFIRVRDGWKKLQLGELIPIPA

SEQ ID NO: 3 - protein, 33 aa, Homo sapiens
VASLRQQVEALQGQVQHLQAAFSQYKKVELFPN

SEQ ID NO: 4 - protein, 31 aa, Homo sapiens
LKSRLDTLAQEVALLKEQQALQTVCLKGTKV

SEQ ID NO: 5 - protein, 82 aa, Homo sapiens
DQTLTEKEKEIEVERVKAESAQASAKMLHEMQRKNEQMMEQKERSYQEHLKQLTEKMENDRVQLLKEQERTLALKLQEQEQL

SEQ ID NO: 6 - protein, 65 aa, Homo sapiens
AKVFSFILVTTALTMGREISALEDCAQEQMRLRAQVRLLETRVKQQQVKIKQLLQENEVQFLDKG

SEQ ID NO: 7 - protein, 740 aa, Homo sapiens
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHAEMGAIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 8 - protein, 244 aa, Homo sapiens
MRWCLLLIWAQGLRQAPLASGMMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLLGAMAATLVVICTAVIVVVALTRKKKALRIHSVEGDLRRKSAGQEEWSPSAPSPPGSCVQAEAAPAGLCGEQRGEDCAELHDYFNVLSYRSLGNCSFFTETG

SEQ ID NO: 9 - protein, 525 aa, Homo sapiens
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 10 - protein, 133 aa, Homo sapiens
MVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 11 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 12 - protein, 250 aa, Homo sapiens [sequence omitted]

SEQ ID NO: 13 - protein, 293 aa, Homo sapiens
MSGLGRSRRGGRSRVDQEERFPQGLWTGVAMRSCPEEQYWDPLLGTCMSCKTICNHQSQRTCAAFCRSLSCRKEQGKFYDHLLRDCISCASICGQHPKQCAYFCENKLRSPVNLPPELRRQRSGEVENNSDNSGRYQGLEHRGSEASPALPGLKLSADQVALVYSTLGLCLCAVLCCFLVAVACFLKKRGDPCSCQPRSRPRQSPAKSSQDHAMEAGSPVSTSPEPVETCSFCFPECRAPTQESAVTPGTPDPTCAGRWGCHTRTTVLQPCPHIPDSGLGIVCVPAQEGGPGA

SEQ ID NO: 14 - protein, 249 aa, Mus musculus
MAMAFCPKDQYWDSSRKSCVSCALTCSQRSQRTCTDFCKFINCRKEQGRYYDHLLGACVSCDSTCTQHPQQCAHFCEKRPRSQANLQPELGRPQAGEVEVRSDNSGRHQGSEHGPGLRLSSDQLTLYCTLGVCLCAIFCCFLVALASFLRRRGEPLPSQPAGPRGSQANSPHAHRPVTEACDEVTASPQPVETCSFCFPERSSPTQESAPRSLGIHGFAGTAAPQPCMRATVGGLGVLRASTGDARPAT

SEQ ID NO: 15 - protein, 181 aa, Homo sapiens
MLLEPGRGCCALAILLAIVDIQSGGCINITSSASQEGTRLNLICTVWHKKEEAEGFVVFLCKDRSGDCSPETSLKQLRLKRDPGIDGVGEISSQLMFTISQVTPLHSGTYQCCARSQKSGIRLQGHFFSILFTETGNYTVTGLKQRQHLEFSHNEGTLSSGFLQEKVWVMLVTSLVALQAL

SEQ ID NO: 16 - protein, 243 aa, Homo sapiens
MCSRGWDSCLALELLLLPLSLLVTSIQGHLVHMTVVSGSNVTLNISESLPENYKQLTWFYTFDQKIVEWDSRKSKYFESKFKGRVRLDPQSGALYISKVQKEDNSTYIMRVLKKTGNEQEWKIKLQVLDPVPKPVIKIEKIEDMDDNCYLKLSCVIPGESVNYTWYGDKRPFPKELQNSVLETTLMPHNYSRCYTCQVSNSVSSKNGTVCLSPPCTLARSFGVEWIASWLVVTVPTILGLLLT

SEQ ID NO: 17 - protein, 187 aa, Homo sapiens
MTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 18 - protein, 205 aa, Homo sapiens
MTPPERLFLPRVCGTTLHLLLLGLLLVLLPGAQGLPGVGLTPSAAQTARQHPKMHLAHSTLKPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFAL

SEQ ID NO: 19 - protein, 352 aa, Homo sapiens
MEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 20 - protein, 166 aa, Homo sapiens
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 21 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 22 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 23 - protein, 93 aa, Homo sapiens
MNAKVVVVLVLVLTALCLSDGKPVSLSYRCPCRFFESHVARANVKHLKILNTPNCALQIVARLKNNNRQVCIDPKLKWIQEYLEKALNKRFKM

SEQ ID NO: 24 - protein, 93 aa, Mus musculus
MDAKVVAVLALVLAALCISDGKPVSLSYRCPCRFFESHIARANVKHLKILNTPNCALQIVARLKNNNRQVCIDPKLKWIQEYLEKALNKRLKM

SEQ ID NO: 25 - protein, 532 aa, Homo sapiens
MAPSSPRPALPALLVLLGALFPGPGNAQTSVSPSKVILPRGGSVLVTCSTSCDQPKLLGIETPLPKKELLLPGNNRKVYELSNVQEDSQPMCYSNCPDGQSTAKTFLTVYWTPERVELAPLPSWQPVGKNLTLRCQVEGGAPRANLTVVLLRGEKELKREPAVGEPAEVTTTVLVRRDHHGANFSCRTELDLRPQGLELFENTSAPYQLQTFVLPATPPQLVSPRVLEVDTQGTVVCSLDGLFPVSEAQVHLALGDQRLNPTVTYGNDSFSAKASVSVTAEDEGTQRLTCAVILGNQSQETLQTVTIYSFPAPNVILTKPEVSEGTEVTVKCEAHPRAKVTLNGVPAQPLGPRAQLLLKATPEDNGRSFSCSATLEVAGQLIHKNQTRELRVLYGPRLDERDCPGNWTWPENSQQTPMCQAWGNPLPELKCLKDGTFPLPIGESVTVTRDLEGTYLCRARSTQGEVTRKVTVNVLSPRYEIVIITVVAAAVIMGTAGLSTYLYNRQRKIKKYRLQQAQKGTPMKPNTQATPP

SEQ ID NO: 26 - protein, 537 aa, Mus musculus
MASTRAKPTLPLLLALVTVVIPGPGDAQVSIHPREAFLPQGGSVQVNCSSSCKEDLSLGLETQWLKDELESGPNWKLFELSEIGEDSSPLCFENCGTVQSSASATITVYSFPESVELRPLPAWQQVGKDLTLRCHVDGGAPRTQLSAVLLRGEEILSRQPVGGHPKDPKEITFTVLASRGDHGANFSCRTELDLRPQGLALFSNVSEARSLRTFDLPATIPKLDTPDLLEVGTQQKLFCSLEGLFPASEARIYLELGGQMPTQESTNSSDSVSATALVEVTEEFDRTLPLRCVLELADQILETQRTLTVYNFSAPVLTLSQLEVSEGSQVTVKCEAHSGSKVVLLSGVEPRPPTPQVQFTLNASSEDHKRSFFCSAALEVAGKFLFKNQTLELHVLYGPRLDETDCLGNWTWQEGSQQTLKCQAWGNPSPKMTCRRKADGALLPIGVVKSVKQEMNGTYVCHAFSSHGNVTRNVYLTVLYHSQNNWTIIILVPVLLVIVGLVMAASYVYNRQRKIRIYKLQKAQEEAIKLKGQAPPP

SEQ ID NO: 27 - protein, 22 aa, Vesiculovirus indiana
IASFFFIIGLIIGLFLVLRVGI

SEQ ID NO: 28 - protein, 37 aa, Vesiculovirus indiana
PIELVEGWFSSWKSSIASFFFIIGLIIGLFLVLRVGI

SEQ ID NO: 29 - protein, 53 aa, Vesiculovirus indiana
DDESLFFGDTGLSKNPIELVEGWFSSWKSSIASFFFIIGLIIGLFLVLRVGIH

SEQ ID NO: 30 - protein, 81 aa, Vesiculovirus indiana
GMLDSDLHLSSKAQVFEHPHIQDAASQLPDDESLFFGDTGLSKNPIELVEGWFSSWKSSIASFFFIIGLIIGLFLVLRVGI

SEQ ID NO: 31 - protein, 25 aa, Vesiculovirus indiana
HLCIKLKHTKKRQIYTDIEMNRLGK

SEQ ID NO: 32 - protein, 23 aa, Influenza A virus
IITIGSVCMTIGMANLILQIGNI

SEQ ID NO: 33 - protein, 23 aa, Influenza A virus
LAIYSTVASSLVLVVSLGAISFW

SEQ ID NO: 34 - protein, 52 aa, Dengue virus
AYGVLFSGVSWTMKIGIGILLTWLGLNSRSTSLSMTCIAVGMVTLYLGVMVQ

SEQ ID NO: 35 - protein, 21 aa, Human immunodeficiency virus 1
FIMIVGGLVGLRIVFAVLSIV